(19) United States
(12) Patent Application Publication
(10) Pub. No.: US 2025/0114396 A1
(43) Pub. Date: Apr. 10, 2025

(54) CHLORITE USES FOR STEM CELLS

(71) Applicant: The Regents of the University of California, Oakland, CA (US)

(72) Inventor: Michael S. McGrath, Meadow Vista, CA (US)

(21) Appl. No.: 18/764,882

(22) Filed: Jul. 5, 2024

Related U.S. Application Data
(60) Provisional application No. 63/512,578, filed on Jul. 7, 2023.

(51) Int. Cl. A61K 33/20; A61K 47/02; A61P 39/00

(52) U.S. Cl. CPC A61K 33/20; A61K 47/02; A61P 39/00

(57) ABSTRACT

Described herein are methods using chlorite formulations to affect stem cell activity.

### CROSS REFERENCE

This present application claims the benefit of U.S. Provisional Application No. 63/512,578 filed on Jul. 7, 2023, which is incorporated herein by reference in its entirety.

### BACKGROUND

Amyotrophic lateral sclerosis (ALS) is a progressive neurodegenerative disease that destroys motor neurons in the brain and spinal cord. ALS eventually leads to muscle paralysis and death, often within 2-4 years of diagnosis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein is a method of reducing a deleterious effect of aging in a subject, the method comprising administering to the subject an effective amount of a chlorite salt, wherein the deleterious effect of aging is not a disease and is not life-threatening.

Disclosed herein is a method comprising: a) administering to a subject an effective amount of a chlorite salt; and b) subsequent to the administering, evaluating telomere length in a stem cell taken from the subject after the administering.

Disclosed herein is a method comprising: a) administering to a subject an effective amount of a chlorite salt; and b) subsequent to the administering, monitoring cell division in a stem cell taken from the subject after the administering.

Disclosed herein is a method comprising: a) administering to a subject an effective amount of a chlorite salt; and b) subsequent to the administering, monitoring the subject for depletion of a telomere of a stem cell of the subject.

Disclosed herein is a method comprising: a) administering to a subject an effective amount of a chlorite salt; and b) subsequent to the administering, monitoring the subject for replication of stem cells.

### BRIEF DESCRIPTION OF THE DRAWINGS

FIG. 1 illustrates the loss of hematopoietic stem cell (HSC) renewal in situations with chronic innate immune system activation.

FIG. 2 illustrates a pulse therapy administration procedure for NP001 consisting a 30 minute infusion 5 days in a row in the first month followed by 3 days in a row per month thereafter.

FIG. 3 illustrates reversal of microbial translocation-associated immune activation by NP001.

FIG. 4 illustrates the effect of NP001 on innate immune activation and dysfunctional immune response.

FIG. 5 illustrates reversal of LPS-activated hematopoietic stem cell (HSC) genetic pathways following a single dose of NP001.

FIG. 6 provides a summary of experimental methods for treatment of HSCs treated with LPS and NP001 and RNAseq analysis.

FIG. 7 summarizes significantly upregulated and downregulated gene pathways in LPS and NP001-treated HSCs.

FIG. 8 summarizes differentially regulated genes in LPS and NP001-treated HSCs.

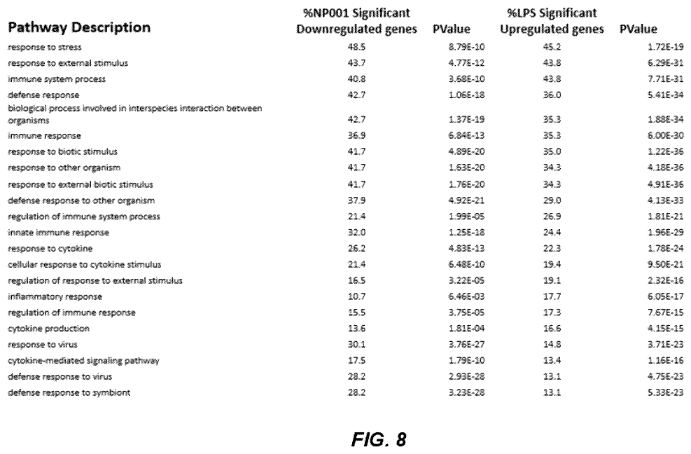

FIG. 9 summarizes factors produced by LPS-activated HSCs that are reversed by treatment with NP001.

FIG. 10 illustrates CCL13-related signaling pathways.

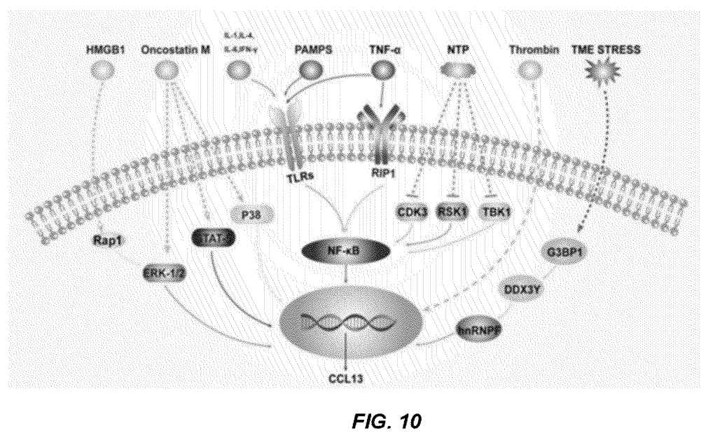

FIG. 11 illustrates CCL13 sources and targets in the innate immune system.

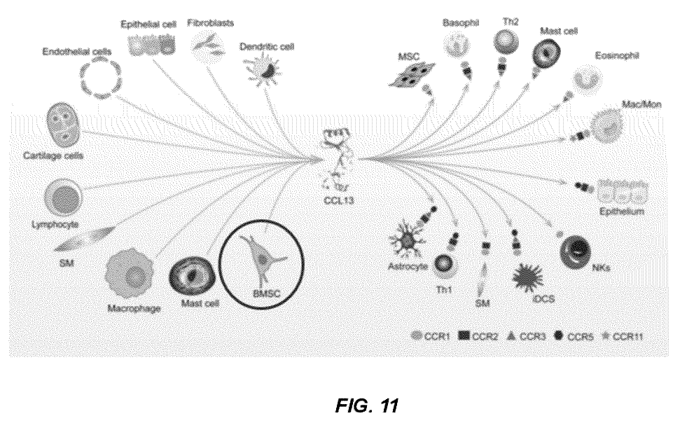

FIG. 12 illustrates CCL7 signaling and disease targets.

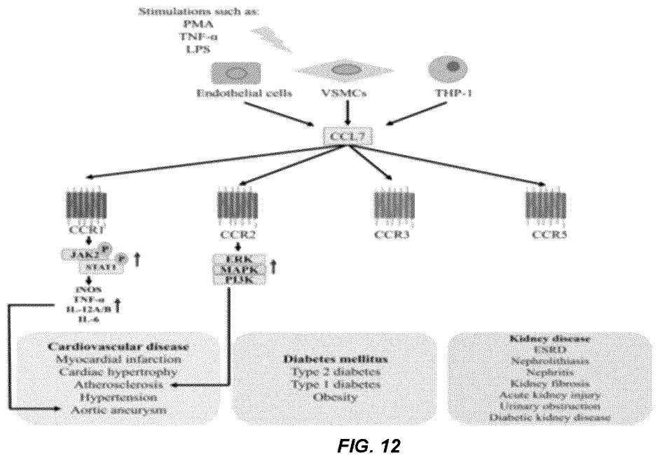

FIG. 13 illustrates genes involved active in MI and M2 macrophages, including CXCL10.

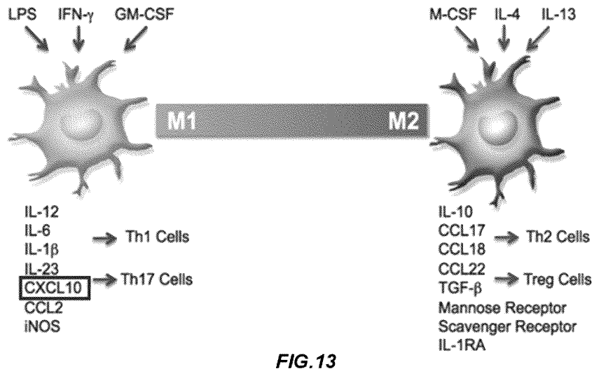

FIG. 14 summarizes chemokine activity across different cell types.

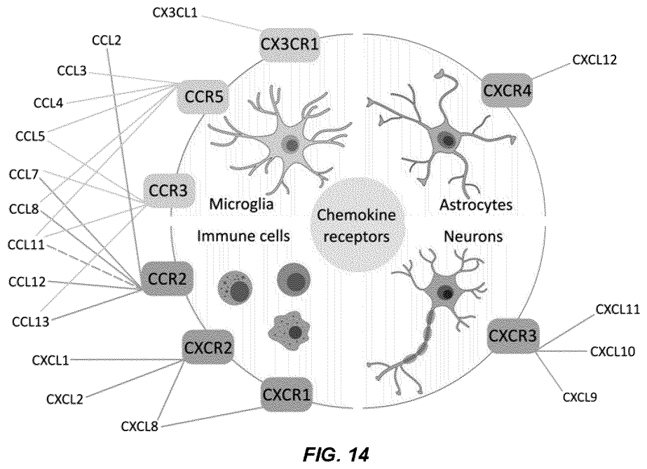

### DETAILED DESCRIPTION OF THE INVENTION

Amyotrophic lateral sclerosis (ALS), also known as Lou Gehrig's disease, is a fatal neurodegenerative disease associated with inappropriate immune system dysfunction involving NF-kB activation, proinflammatory factor production, and progressive changes in motor neuron function. Factors elaborated by spinal cord microglia both damage and inhibit repair of neurons injured by the accumulation of misfolded proteins. Inflammation and immune system dysregulation are involved in the progression of ALS, including the presence of activated macrophages in ALS patients. Compounds that can reduce inflammation and immune system dysregulation, possibly by returning macrophages to their inactivated state, can be effective in treating ALS or other disorders with immune system dysfunction.

Macrophages are white blood cells produced by the division of monocytes, which play a role in innate immunity (non-specific immune defenses) and help to initiate adaptive immunity (specific defense mechanisms). The cells phagocytose (i.e., engulf and then digest) cellular debris and pathogens either as stationary or as mobile cells. When activated by pathogens or other mechanisms, macrophages stimulate and recruit lymphocytes and other immune cells to respond. Activated macrophages are involved in the progression of several diseases and disorders, including amyotrophic lateral sclerosis (ALS). Activated macrophages elicit massive leukocyte infiltration and flood surrounding tissue with inflammatory mediators, pro-apoptotic factors, and matrix degrading proteases. These actions can result in inflammation that can dismantle tissues to the point of inflicting serious injury. Tissue destruction perpetrated by macrophage-induced inflammation, a form of immune system dysregulation, can be associated with the progression of the degenerative disease ALS.

ALS patients can be defined by four generalized patient groups: slowly progressive, fast progressive, early disease presentation, and late disease presentation. Of the four categories, only the slowly progressive patient group includes inflammation as a significant aspect of ALS disease pathology. The slowly progressive subset's association with inflammation is inferred upon study of ALS in association with C-reactive protein (CRP) levels over time from diagnosis. The longer an ALS patient lives after diagnosis, the higher the plasma CRP. A large subgroup of ALS patients is evidence for ongoing. and potentially growing. inflammation associated with a slower rate of progression. Methods of identifying ALS patients with inflammation are needed to treat such ALS patients with targeted immune regulating therapies.

ALS can also be distinguished by ALS symptoms, genetic cause, lack of clear genetic association, or combinations thereof. In comes embodiments, subjects with ALS can be categorized as sporadic or inherited ALS. Sporadic ALS pathology occurs in subjects with no genetic or family history of ALS, while inherited ALS pathology occurs in subjects with a genetic or family history of ALS. As many as 90% to 95% of ALS cases can be sporadic and occur in subjects with no genetic or family history of ALS.

Mutations to one or more genes can be associated with subjects experiencing either an inherited or sporadic ALS pathology. For instance, 60% of individuals with familial ALS can have an identified genetic mutation to one or more genes associated with the condition. In some embodiments, subjects experiencing either an inherited ALS pathology have inherited mutations to one or more of the genes listed in TABLE 1. Mutations in the C9orf72 gene account for 30% to 40% of inherited ALS in the United States and Europe; worldwide SOD1 gene mutations cause 15% to 20% of inherited ALS; and TARDBP and FUS gene mutations each account for about 5% of cases of inherited ALS.

In some embodiments, mutations to one or more genes listed in TABLE 1 can be important to the normal functioning of motor neurons and other cells, and mutations to one or more of these genes can contribute to the decline in function or death of motor neurons in ALS patients. In some embodiments, mutations to one or more genes of TABLE 1 can contribute to a decline in motor neutron function or neuron death as a result of buildup of protein aggregates in motor neurons, a slowing in the transport of materials needed for the proper function of axons in motor neurons, an accumulation of toxic substances in the motor neurons, or combinations thereof.

TABLE 1
Gene Associated with ALS

| | |
|---|---|
| 1 | SOD1 |
| 2 | ALS2 |
| 3 | Chromosome 18 |
| 4 | SEXT |
| 5 | SPG11 |
| 6 | FUS |
| 7 | Chromosome 20 |
| 8 | VAPB |
| 9 | ANG |
| 10 | TARDBP/TDP-43 |
| 11 | FIG4 |
| 12 | OPTN |
| 13 | ATXN2 |
| 14 | VCP |
| 15 | UBQLN2 |
| 16 | SIGMAR1 |
| 17 | CHMP2B |
| 18 | PFN1 |
| 19 | ERBB4 |
| 20 | HNRNPA1 |
| 21 | MATR3 |
| 22 | TUBA4A |
| 23 | ANXA11 |
| 24 | NEK1 |
| 25 | C9orf72 |
| 26 | CHCHD10 |
| 27 | SQSTM1 |
| 28 | TARDBP |
| 29 | SETX |
| 30 | TAF15 |
| 31 | EWSR1 |
| 32 | hnRNPA2B1 |
| 33 | ELP3 |
| 34 | TBK1 |
| 35 | DCTN1 |
| 36 | NEFH |
| 37 | PRPH |
| 38 | C19ORF12 |
| 39 | SS18L1 |
| 40 | PNPLA6 |
| 41 | PON1-3 |
| 42 | DAO |
| 43 | CHRNA3, 4, B4 |
| 44 | ALS3 |
| 45 | ALS7 |
| 46 | ALS6-21 |
| 47 | ALS-FTD |

Initial events of ALS pathogenesis occur at the neuromuscular junction where neuron axonal processes interact with muscle out-side the central nervous system. This reaction is inflammatory and is mediated by components of the innate immune system including acute phase reactant proteins and blood derived granulocytes and macrophages. The triggering events can be linked to the presence of abnormally folded or aggregated proteins associated with ALS such as TDP43 and SOD-1, recognized by the innate immune system within the neuromuscular junction. Normally, the acute phase reaction is followed closely by immune signals that turn off that reaction to have the immune system remain in balance. Therefore, the plasma based or humoral innate immune system provides feedback on activated macrophages, the cellular drivers of inflammation and balance the inflammatory with anti-inflammatory immune signals. Proteins associated with the acute phase innate immune response can be measured in the blood and one, C-reactive protein (CRP), allows quantitative determination of the degree of inflammation associated with disease.

Plasma CRP levels are acutely elevated more than 30-fold in an acute phase reaction, but rapidly fall as the initiator of the inflammation becomes controlled. Two categories of insult cause the synthesis of CRP predominantly from the liver: infection and tissue damage. Infections can trigger both the innate and the adaptive response so that blood levels of factors produced by both immune reactions are present in the plasma. Although CRP rapidly appears after an insult and is viewed as a marker for inflammation severity, the general function is to facilitate phagocytic cell clearance of foreign material. In addition, CRP down regulates other proinflammatory components of the innate immune response to turn down the production of inflammatory by-products. Therefore, if CRP levels are chronically elevated, then ongoing infectious or tissue damaging processes are possibly driving the persistent CRP response. Continued tissue damage absent the persistence of an acute phase process suggests uncoupling of the innate immune response to signals that normally drive that reaction.

Other plasma factors involved in innate immune response include serum amyloid A (SAA), levels of which increase in parallel with CRP. SAA binds to and clears bacterial byproducts such as lipopolysaccharide (LPS). Alpha 2 macroglobulin (A2M) becomes activated when the acute phase reaction is initiated and clears by-products of damaged cells, specifically proteases. When further activated by hypochlorite, a byproduct of the oxidative burst reaction initiated by phagocyte (granulocytes, macrophages) activation, A2M forms a dimer, releases preformed TGFB1, and binds to and removes misfolded proteins and aggregates.

The innate immune system responds rapidly to infection and/or tissue damage. Within minutes, the acute phase reactants CRP and serum amyloid A (SAA) increase more than 30-fold in plasma. When an inflammatory response is initiated, byproducts of that response need to be removed or neutralized to avoid significant tissue damage. CRP facilitates the clearance of dead cells and protein aggregates. SAA binds to and removes products of bacterial clearance mediated by activated phagocytes such as LPS.

TGFB1 is a potent regulator of inflammation and down regulates inflammatory drive. Together, CRP, SAA, and A2M represent major humoral components of the innate immune system and coordinate to regulate the degree of inflammatory reactions associated with the activated cellular components of the response (macrophages, granulocytes).

NP001 can augment the innate immune activation cycle in ALS patients with elevated baseline plasma CRP. The innate immune activation cycle is a self-regulated process that occurs after the immune system is exposed to infection or tissue damage. The initiator of immune system activation can be the presence of misfolded or aggregated proteins, including TDP43.

In response to immune system activation, blood derived macrophages undergo oxidative burst and release hypochlorous acid as a byproduct. The acute phase response involves elevation of CRP and SAA. CRP facilitates clearance of damaged cells and tissues. SAA binds to and clears bacterial byproducts, including LPS. A2M is activated to remove damaged cell products, including proteases. Hypochlorous acid stimulates the production of taurine chloramine (TauCl) and promotes dimerization of A2M. A2M dimers clear misfolded proteins and release pre-synthesized TGFB1 to feedback on proinflammatory cells and turn off NFkB. NP001 is converted in vivo to HClO and stimulates dimerization of A2M and subsequent release of pre-synthesized TGFB1.

### Methods

Disclosed herein are methods of identifying a subject with ALS that can be responsive to immune regulating therapies. In some embodiments, disclosed herein is a method of treating a subject with slowly progressive ALS with a pharmaceutical composition comprising sodium chlorite. In some embodiments, disclosed herein is a pharmaceutical composition for use in treating a condition, for example, ALS, Parkinson's disease, Alzheimer's disease, chronic obstructive pulmonary disease (COPD), or colitis.

Disclosed herein are compositions and methods for treating neurodegenerative diseases. Non-limiting examples of neurodegenerative diseases include amyotrophic lateral sclerosis (ALS, or Lou Gehrig's disease), Alzheimer's disease, vascular dementia, Parkinson's disease, multiple sclerosis, and primary progressive multiple sclerosis. In some embodiments, the neurodegenerative disease is ALS. In some embodiments, the neurodegenerative disease is Alzheimer's disease. In some embodiments, the neurodegenerative disease is Parkinson's disease. Treatments can include, for example, a formulation of sodium chlorite.

A subject for a therapy herein can be of any suitable age, for example, at least 30 years old, at least 35 years old, at least 40 years old, at least 45 years old, at least 50 years old, at least 55 years old, at least 60 years old, 40 to 80 years old, 40 to 75 years old, 40 to 70 years old, 40 to 65 years old, 40 to 60 years old, 40 to 55 years old, 40 to 50 years old, no greater than 80 years old, no greater than 75 years old, no greater than 70 years old, no greater than 65 years old, or no greater than 80 years old.

A subject described herein can be of any suitable age. In some embodiments, the subject is at least age 40, at least age 41, at least age 42, at least age 43, at least age 44, at least age 45, at least age 46, at least age 47, at least age 48, at least age 49, at least age 50, at least age 51, at least age 52, at least age 53, at least age 54, at least age 55, at least age 56, at least age 57, at least age 58, at least age 59, at least age 60, at least age 61, at least age 62, at least age 63, at least age 64, or at least age 65. In some embodiments, the subject is at least age 40, at least age 41, at least age 42, at least age 43, at least age 44, at least age 45, at least age 46, at least age 47, at least age 48, at least age 49, or at least age 50. In some embodiments, the subject is at least age 51, at least age 52, at least age 53, at least age 54, at least age 55, at least age 56, at least age 57, at least age 58, at least age 59, at least age 60, at least age 61, at least age 62, at least age 63, at least age 64, or at least age 65. In some embodiments, the subject is at least age 40. In some embodiments, the subject is at least age 41. In some embodiments, the subject is at least age 42. In some embodiments, the subject is at least age 43. In some embodiments, the subject is at least age 44. In some embodiments, the subject is at least age 44. In some embodiments, the subject is at least age 45. In some embodiments, the subject is at least age 46. In some embodiments, the subject is at least age 47. In some embodiments, the subject is at least age 48. In some embodiments, the subject is at least age 49. In some embodiments, the subject is at least age 50. In some embodiments, the subject is at least age 51. In some embodiments, the subject is at least age 52. In some embodiments, the subject is at least age 53. In some embodiments, the subject is at least age 54. In some embodiments, the subject is at least age 55. In some embodiments, the subject is at least age 56. In some embodiments, the subject is at least age 57. In some embodiments, the subject is at least age 58. In some embodiments, the subject is at least age 59. In some embodiments, the subject is at least age 60. In some embodiments, the subject is at least age 61. In some embodiments, the subject is at least age 62. In some embodiments, the subject is at least age 63. In some embodiments, the subject is at least age 64. In some embodiments, the subject is at least age 65.

In some embodiments, the subject is no greater than age 40, no greater than age 41, no greater than age 42, no greater than age 43, no greater than age 44, no greater than age 45, no greater than age 46, no greater than age 47, no greater than age 48, no greater than age 49, no greater than age 50, no greater than age 51, no greater than age 52, no greater than age 53, no greater than age 54, no greater than age 55, no greater than age 56, no greater than age 57, no greater than age 58, no greater than age 59, no greater than age 60, no greater than age 61, no greater than age 62, no greater than age 63, no greater than age 64, or no greater than age 65.

In some embodiments, the subject is no greater than age 41, no greater than age 42, no greater than age 43, no greater than age 44, no greater than age 45, no greater than age 46, no greater than age 47, no greater than age 48, no greater than age 49, or no greater than age 50. In some embodiments, the subject is no greater than age 51, no greater than age 52, no greater than age 53, no greater than age 54, no greater than age 55, no greater than age 56, no greater than age 57, no greater than age 58, no greater than age 59, no greater than age 60, no greater than age 61, no greater than age 62, no greater than age 63, no greater than age 64, or no greater than age 65. In some embodiments, the subject is no greater than age 40. In some embodiments, the subject is no greater than age 41. In some embodiments, the subject is no greater than age 42. In some embodiments, the subject is no greater than age 43. In some embodiments, the subject is no greater than age 44. In some embodiments, the subject is no greater than age 44. In some embodiments, the subject is no greater than age 45. In some embodiments, the subject is no greater than age 46. In some embodiments, the subject is no greater than age 47. In some embodiments, the subject is no greater than age 48. In some embodiments, the subject is no greater than age 49. In some embodiments, the subject is no greater than age 50. In some embodiments, the subject is no greater than age 51. In some embodiments, the subject is no greater than age 52. In some embodiments, the subject is no greater than age 53. In some embodiments, the subject is no greater than age 54. In some embodiments, the subject is no greater than age 55. In some embodiments, the subject is no greater than age 56. In some embodiments, the subject is no greater than age 57. In some embodiments, the subject is no greater than age 58. In some embodiments, the subject is no greater than age 59. In some embodiments, the subject is no greater than age 60. In some embodiments, the subject is no greater than age 61. In some embodiments, the subject is no greater than age 62. In some embodiments, the subject is no greater than age 63. In some embodiments, the subject is no greater than age 64. In some embodiments, the subject is no greater than age 65.

In some embodiments, the subject is about age 40, about age 41, about age 42, about age 43, about age 44, about age 45, about age 46, about age 47, about age 48, about age 49, about age 50, about age 51, about age 52, about age 53, about age 54, about age 55, about age 56, about age 57, about age 58, about age 59, about age 60, about age 61, about age 62, about age 63, about age 64, or about age 65. In some embodiments, the subject is about age 40, about age 41, about age 42, about age 43, about age 44, about age 45, about age 46, about age 47, about age 48, about age 49, or about age 50. In some embodiments, the subject is about age 51, about age 52, about age 53, about age 54, about age 55, about age 56, about age 57, about age 58, about age 59, about age 60, about age 61, about age 62, about age 63, about age 64, or about age 65. In some embodiments, the subject is about age 40. In some embodiments, the subject is about age 41. In some embodiments, the subject is about age 42. In some embodiments, the subject is about age 43. In some embodiments, the subject is about age 44. In some embodiments, the subject is about age 44. In some embodiments, the subject is about age 45. In some embodiments, the subject is about age 46. In some embodiments, the subject is about age 47. In some embodiments, the subject is about age 48. In some embodiments, the subject is about age 49. In some embodiments, the subject is about age 50. In some embodiments, the subject is about age 51. In some embodiments, the subject is about age 52. In some embodiments, the subject is about age 53. In some embodiments, the subject is about age 54. In some embodiments, the subject is about age 55. In some embodiments, the subject is about age 56. In some embodiments, the subject is about age 57. In some embodiments, the subject is about age 58. In some embodiments, the subject is about age 59. In some embodiments, the subject is about age 60. In some embodiments, the subject is about age 61. In some embodiments, the subject is about age 62. In some embodiments, the subject is about age 63. In some embodiments, the subject is about age 64. In some embodiments, the subject is about age 65.

In some embodiments, the subject has a neurodegenerative disease. In some embodiments, the subject has ALS. In some embodiments, the subject has Alzheimer's disease. In some embodiments, the subject has Parkinson's disease.

In some embodiments, the subject possesses a CRP level greater than 1.13 mg/L as determined by a blood assay. In some embodiments, the subject possesses a CRP level greater than 1.15 mg/L as determined by a blood assay. In some embodiments, the subject possesses a CRP level greater than 1.17 mg/L as determined by a blood assay. In some embodiments, the subject possesses a CRP level greater than 1.2 mg/L as determined by a blood assay. In some embodiments, the subject possesses a CRP level greater than 1.5 mg/L as determined by a blood assay. In some embodiments, the subject possesses a CRP level greater than 2 mg/L as determined by a blood assay. In some embodiments, the subject possesses a CRP level greater than 2.5 mg/L as determined by a blood assay. In some embodiments, the subject possesses a CRP level greater than 3 mg/L as determined by a blood assay. In some embodiments, the subject possesses a CRP level greater than 3.5 mg/L as determined by a blood assay. In some embodiments, the subject possesses a CRP level greater than 4 mg/L as determined by a blood assay.

In some embodiments, the subject is administered an amount of sodium chlorite. In some embodiments, the subject is administered an amount of sodium chlorite that is therapeutically effective for ALS.

In some embodiments, the amount is about 0.2 mg/kg/day to about 3.5 mg/kg/day. In some embodiments, the amount is about 0.5 mg/kg/day to about 3.5 mg/kg/day. In some embodiments, the amount is about 1.0 mg/kg/day to about 3.5 mg/kg/day. In some embodiments, the amount is about 1.5 mg/kg/day to about 3.5 mg/kg/day. In some embodiments, the amount is about 2.0 mg/kg/day to about 3.5 mg/kg/day. In some embodiments, the amount is about 2.5 mg/kg/day to about 3.5 mg/kg/day.

In some embodiments, the amount is about 1.0 mg/kg/day to about 10 mg/kg/day. In some embodiments, the amount is about 2.0 mg/kg/day to about 10 mg/kg/day. In some embodiments, the amount is about 3.0 mg/kg/day to about 10 mg/kg/day. In some embodiments, the amount is about 4.0 mg/kg/day to about 10 mg/kg/day. In some embodiments, the amount is about 5.0 mg/kg/day to about 10 mg/kg/day.

In some embodiments, the amount is about 0.2 mg/kg/day. In some embodiments, the amount is about 0.5 mg/kg/day. In some embodiments, the amount is about 1.0 mg/kg/day. In some embodiments, the amount is about 1.2 mg/kg/day. In some embodiments, the amount is about 1.4 mg/kg/day. In some embodiments, the amount is about 1.6 mg/kg/day. In some embodiments, the amount is about 1.8 mg/kg/day. In some embodiments, the amount is about 2.0 mg/kg/day. In some embodiments, the amount is about 2.2 mg/kg/day. In some embodiments, the amount is about 2.4 mg/kg/day. In some embodiments, the amount is about 2.6 mg/kg/day. In some embodiments, the amount is about 2.8 mg/kg/day. In some embodiments, the amount is about 3.0 mg/kg/day. In some embodiments, the amount is about 3.2 mg/kg/day. In some embodiments, the amount is about 3.5 mg/kg/day.

In some embodiments, the amount is about 4.0 mg/kg/day. In some embodiments, the amount is about 5.0 mg/kg/day. In some embodiments, the amount is about 6.0 mg/kg/day. In some embodiments, the amount is about 7.0 mg/kg/day. In some embodiments, the amount is about 8.0 mg/kg/day. In some embodiments, the amount is about 9.0 mg/kg/day. In some embodiments, the amount is about 10.0 mg/kg/day.

In some embodiments, the administering is oral. In some embodiments, the administering is parenteral. In some embodiments, the administering is intravenous.

In some embodiments, the subject is on a regimen of sodium chlorite. In some embodiments, the regimen of sodium chlorite is about 0.2 mg/kg/day to about 3.5 mg/kg/day.

In some embodiments, the regimen of sodium chlorite is about 0.2 mg/kg/day to about 3.5 mg/kg/day. In some embodiments, the regimen of sodium chlorite is about 0.5 mg/kg/day to about 3.5 mg/kg/day. In some embodiments, the regimen of sodium chlorite is about 1.0 mg/kg/day to about 3.5 mg/kg/day. In some embodiments, the regimen of sodium chlorite is about 1.5 mg/kg/day to about 3.5 mg/kg/day. In some embodiments, the regimen of sodium chlorite is about 2.0 mg/kg/day to about 3.5 mg/kg/day. In some embodiments, the regimen of sodium chlorite is about 2.5 mg/kg/day to about 3.5 mg/kg/day.

In some embodiments, the regimen of sodium chlorite is about 1.0 mg/kg/day to about 10.0 mg/kg/day. In some embodiments, the regimen of sodium chlorite is about 2.0 mg/kg/day to about 10.0 mg/kg/day. In some embodiments, the regimen of sodium chlorite is about 3.0 mg/kg/day to about 10.0 mg/kg/day. In some embodiments, the regimen of sodium chlorite is about 4.0 mg/kg/day to about 10.0 mg/kg/day. In some embodiments, the regimen of sodium chlorite is about 5.0 mg/kg/day to about 10.0 mg/kg/day.

In some embodiments, the regimen of sodium chlorite is about 0.2 mg/kg/day. In some embodiments, the regimen of sodium chlorite is about 0.5 mg/kg/day. In some embodiments, the regimen of sodium chlorite is about 1.0 mg/kg/day. In some embodiments, the regimen of sodium chlorite is about 1.2 mg/kg/day. In some embodiments, the regimen of sodium chlorite is about 1.4 mg/kg/day. In some embodiments, the regimen of sodium chlorite is about 1.6 mg/kg/day. In some embodiments, the regimen of sodium chlorite is about 1.8 mg/kg/day. In some embodiments, the regimen of sodium chlorite is about 2.0 mg/kg/day. In some embodiments, the regimen of sodium chlorite is about 2.2 mg/kg/day. In some embodiments, the regimen of sodium chlorite is about 2.4 mg/kg/day. In some embodiments, the regimen of sodium chlorite is about 2.6 mg/kg/day. In some embodiments, the regimen of sodium chlorite is about 2.8 mg/kg/day. In some embodiments, the regimen of sodium chlorite is about 3.0 mg/kg/day. In some embodiments, the regimen of sodium chlorite is about 3.2 mg/kg/day. In some embodiments, the regimen of sodium chlorite is about 3.5 mg/kg/day.

In some embodiments, the regimen of sodium chlorite is about 4.0 mg/kg/day. In some embodiments, the regimen of sodium chlorite is about 5.0 mg/kg/day. In some embodiments, the regimen of sodium chlorite is about 6.0 mg/kg/day. In some embodiments, the regimen of sodium chlorite is about 7.0 mg/kg/day. In some embodiments, the regimen of sodium chlorite is about 8.0 mg/kg/day. In some embodiments, the regimen of sodium chlorite is about 9.0 mg/kg/day. In some embodiments, the regimen of sodium chlorite is about 10.0 mg/kg/day.

In some embodiments, the regimen of sodium chlorite is administered orally. In some embodiments, the regimen of sodium chlorite is administered parenterally. In some embodiments, the regimen of sodium chlorite is administered intravenously.

In some embodiments, the subject is undergoing a treatment for ALS, wherein the treatment is a therapeutic regimen of sodium chlorite. In some embodiments, the method further comprises determining to discontinue the therapeutic regimen of sodium chlorite. In some embodiments, the method further comprises determining to continue the therapeutic regimen of sodium chlorite.

In some embodiments, the therapeutic regimen of sodium chlorite is about 0.2 mg/kg/day to about 3.5 mg/kg/day. In some embodiments, the therapeutic regimen of sodium chlorite is about 0.2 mg/kg/day to about 3.5 mg/kg/day. In some embodiments, the therapeutic regimen of sodium chlorite is about 0.5 mg/kg/day to about 3.5 mg/kg/day. In some embodiments, the therapeutic regimen of sodium chlorite is about 1.0 mg/kg/day to about 3.5 mg/kg/day. In some embodiments, the therapeutic regimen of sodium chlorite is about 1.5 mg/kg/day to about 3.5 mg/kg/day. In some embodiments, the therapeutic regimen of sodium chlorite is about 2.0 mg/kg/day to about 3.5 mg/kg/day. In some embodiments, the therapeutic regimen of sodium chlorite is about 2.5 mg/kg/day to about 3.5 mg/kg/day.

In some embodiments, the therapeutic regimen of sodium chlorite is about 1.0 mg/kg/day to about 10.0 mg/kg/day. In some embodiments, the therapeutic regimen of sodium chlorite is about 2.0 mg/kg/day to about 10.0 mg/kg/day. In some embodiments, the therapeutic regimen of sodium chlorite is about 3.0 mg/kg/day to about 10.0 mg/kg/day. In some embodiments, the therapeutic regimen of sodium chlorite is about 4.0 mg/kg/day to about 10.0 mg/kg/day. In some embodiments, the therapeutic regimen of sodium chlorite is about 5.0 mg/kg/day to about 10.0 mg/kg/day.

In some embodiments, the therapeutic regimen of sodium chlorite is about 0.2 mg/kg/day. In some embodiments, the therapeutic regimen of sodium chlorite is about 0.5 mg/kg/day. In some embodiments, the therapeutic regimen of sodium chlorite is about 1.0 mg/kg/day. In some embodiments, the therapeutic regimen of sodium chlorite is about 1.2 mg/kg/day. In some embodiments, the therapeutic regimen of sodium chlorite is about 1.4 mg/kg/day. In some embodiments, the therapeutic regimen of sodium chlorite is about 1.6 mg/kg/day. In some embodiments, the therapeutic regimen of sodium chlorite is about 1.8 mg/kg/day. In some embodiments, the therapeutic regimen of sodium chlorite is about 2.0 mg/kg/day. In some embodiments, the therapeutic regimen of sodium chlorite is about 2.2 mg/kg/day. In some embodiments, the therapeutic regimen of sodium chlorite is about 2.4 mg/kg/day. In some embodiments, the therapeutic regimen of sodium chlorite is about 2.6 mg/kg/day. In some embodiments, the therapeutic regimen of sodium chlorite is about 2.8 mg/kg/day. In some embodiments, the therapeutic regimen of sodium chlorite is about 3.0 mg/kg/day. In some embodiments, the therapeutic regimen of sodium chlorite is about 3.2 mg/kg/day. In some embodiments, the therapeutic regimen of sodium chlorite is about 3.5 mg/kg/day.

In some embodiments, the therapeutic regimen of sodium chlorite is about 4.0 mg/kg/day. In some embodiments, the therapeutic regimen of sodium chlorite is about 5.0 mg/kg/day. In some embodiments, the therapeutic regimen of sodium chlorite is about 6.0 mg/kg/day. In some embodiments, the therapeutic regimen of sodium chlorite is about 7.0 mg/kg/day. In some embodiments, the therapeutic regimen of sodium chlorite is about 8.0 mg/kg/day. In some embodiments, the therapeutic regimen of sodium chlorite is about 9.0 mg/kg/day. In some embodiments, the therapeutic regimen of sodium chlorite is about 10.0 mg/kg/day.

In some embodiments, the therapeutic regimen of sodium chlorite is administered orally. In some embodiments, the therapeutic regimen of sodium chlorite is administered parenterally. In some embodiments, the therapeutic regimen of sodium chlorite is administered intravenously. In some embodiments, the therapeutic regimen of sodium chlorite is administered via intravenous infusion. In some embodiments, the therapeutic regimen of sodium chlorite is administered by intravenous infusion over about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 90 minutes, about 120 minutes, about 150 minutes, or about 180 minutes. In some embodiments, the therapeutic regimen of sodium chlorite is administered by intravenous infusion over about 30 minutes to about 60 minutes. In some embodiments, the therapeutics regimen of sodium chlorite is administered by intravenous infusion over about 30 minutes.

In some embodiments, the subject is undergoing sodium chlorite therapy. In some embodiments, the sodium chlorite therapy is about 0.2 mg/kg/day to about 3.5 mg/kg/day. In some embodiments, the sodium chlorite therapy is about 0.2 mg/kg/day to about 3.5 mg/kg/day. In some embodiments, the sodium chlorite therapy is about 0.2 mg/kg/day to about 3.5 mg/kg/day. In some embodiments, the sodium chlorite therapy is about 0.5 mg/kg/day to about 3.5 mg/kg/day. In some embodiments, the sodium chlorite therapy is about 1.0 mg/kg/day to about 3.5 mg/kg/day. In some embodiments, the sodium chlorite therapy is about 1.5 mg/kg/day to about 3.5 mg/kg/day. In some embodiments, the sodium chlorite therapy is about 2.0 mg/kg/day to about 3.5 mg/kg/day. In some embodiments, the sodium chlorite therapy is about 2.5 mg/kg/day to about 3.5 mg/kg/day.

In some embodiments, the sodium chlorite therapy is about 1.0 mg/kg/day to about 10.0 mg/kg/day. In some embodiments, the sodium chlorite therapy is about 2.0 mg/kg/day to about 10.0 mg/kg/day. In some embodiments, the sodium chlorite therapy is about 3.0 mg/kg/day to about 10.0 mg/kg/day. In some embodiments, the sodium chlorite therapy is about 4.0 mg/kg/day to about 10.0 mg/kg/day. In some embodiments, the sodium chlorite therapy is about 5.0 mg/kg/day to about 10.0 mg/kg/day.

In some embodiments, the sodium chlorite therapy is about 0.2 mg/kg/day. In some embodiments, the sodium chlorite therapy is about 0.5 mg/kg/day. In some embodiments, the sodium chlorite therapy is about 1.0 mg/kg/day. In some embodiments, the sodium chlorite therapy is about 1.2 mg/kg/day. In some embodiments, the sodium chlorite therapy is about 1.4 mg/kg/day. In some embodiments, the sodium chlorite therapy is about 1.6 mg/kg/day. In some embodiments, the sodium chlorite therapy is about 1.8 mg/kg/day. In some embodiments, the sodium chlorite therapy is about 2.0 mg/kg/day. In some embodiments, the sodium chlorite therapy is about 2.2 mg/kg/day. In some embodiments, the sodium chlorite therapy is about 2.4 mg/kg/day. In some embodiments, the sodium chlorite therapy is about 2.6 mg/kg/day. In some embodiments, the sodium chlorite therapy is about 2.8 mg/kg/day. In some embodiments, the sodium chlorite therapy is about 3.0 mg/kg/day. In some embodiments, the sodium chlorite therapy is about 3.2 mg/kg/day. In some embodiments, the sodium chlorite therapy is about 3.5 mg/kg/day.

In some embodiments, the sodium chlorite therapy is about 4.0 mg/kg/day. In some embodiments, the sodium chlorite therapy is about 5.0 mg/kg/day. In some embodiments, the sodium chlorite therapy is about 6.0 mg/kg/day. In some embodiments, the sodium chlorite therapy is about 7.0 mg/kg/day. In some embodiments, the sodium chlorite therapy is about 8.0 mg/kg/day. In some embodiments, the sodium chlorite therapy is about 9.0 mg/kg/day. In some embodiments, the sodium chlorite therapy is about 10.0 mg/kg/day.

In some embodiments, the sodium chlorite therapy can be administered to a subject in an amount based on the body mass of the subject. In some embodiments, the sodium chlorite therapy is about 0.1 mg/kg subject body mass. In some embodiments, the sodium chlorite therapy is about 0.25 mg/kg subject body mass. In some embodiments, the sodium chlorite therapy is about 0.5 mg/kg subject body mass. In some embodiments, the sodium chlorite therapy is about 0.75 mg/kg subject body mass. In some embodiments, the sodium chlorite therapy is about 1.0 mg/kg subject body mass. In some embodiments, the sodium chlorite therapy is about 1.25 mg/kg subject body mass. In some embodiments, the sodium chlorite therapy is about 1.5 mg/kg subject body mass. In some embodiments, the sodium chlorite therapy is about 1.75 mg/kg subject body mass. In some embodiments, the sodium chlorite therapy is about 2 mg/kg subject body mass. In some embodiments, the sodium chlorite therapy is about 2.5 mg/kg subject body mass. In some embodiments, the sodium chlorite therapy is about 3 mg/kg subject body mass. In some embodiments, the sodium chlorite therapy is about 4 mg/kg subject body mass.

In some embodiments, the method further comprises administering to the subject the sodium chlorite therapy. In some embodiments, the method further comprises orally administering to the subject the sodium chlorite therapy. In some embodiments, the method further comprises parenterally administering to the subject the sodium chlorite therapy. In some embodiments, the method further comprises intravenously administering to the subject the sodium chlorite therapy.

A subject can be monitored for biomarkers for a condition prior to, during, or after receiving therapy for the neurodegenerative disease. For example, a subject who received, is receiving, or is recommended to receive sodium chlorite therapy for ALS can be monitored for biomarkers associated with the condition and the therapy.

Non-limiting examples of biomarkers relevant to the therapies herein include liposaccharide (LPS) and wound healing epidermal growth factor (EGF). Monitoring the levels of these biomarkers in a subject, for example, by HPLC analysis of blood draws, can provide information useful for therapeutic decisions. For example, a decision to begin therapy, continue therapy, or end therapy can be based on monitoring the levels of biomarkers in a subject, and by evaluating the ratio of the levels of the biomarkers in the subject over time. A decreasing ratio of LPS:EGF over time can be associated with successful therapy.

In some embodiments, the biomarker is transforming growth factor beta receptor 1 (TGFB1). In some embodiments, TGFB1 acts as a surrogate marker for alpha 2 macroglobulin (A2M) activation into a dimeric form. In some embodiments, the biomarker regulates the innate immune system.

In some embodiments, administration of a compound or formulation herein provides an anti-aging effect. In some embodiments, administration of a compound or formulation herein can reduce deleterious effects of aging. In some embodiments, the deleterious effect of aging is not a disease. In some embodiments, the deleterious effect of aging is not life-threatening. Non-limiting examples of deleterious effects of aging include hearing loss, cataracts and refractive errors, pain (e.g., neck and back pain), osteoarthritis, depression, and sensory deficits. Reduction of deleterious effects of aging in a subject can be determined by observation of the subject. In some embodiments, the deleterious effect of aging is associated with over-replication of stem cells. The anti-aging effect can arise, for example, by reducing the unnecessary, over-replication of stem cells. The reduction of deleterious effects of aging can arise, for example, by reducing the unnecessary, over-replication of stem cells. Over-reproduction results in depletion of the telomeres of the stem cells faster than is needed or desirable to sustain the life and vitality of the organism. Blocking pathways that promote unnecessary replication of stem cells slows the aging process of the stem cells so the cells remain available to heal the organism and slow harmful effects of aging. Results of such therapy can be monitored, for example, by monitoring cell division in a stem cell, monitoring replication of stem cells, monitoring telomere depletion in a stem cell, or by quantitative evaluation of telomere length in a subject to monitor telomere shortening or lack thereof. In some embodiments, monitoring of cell division in a stem cell can determine that cell division occurs at a rate that is lesser than what the rate of cell division would have been had a subject not received a compound or formulation herein. In some embodiments, monitoring of replication of stem cells can determine that replication occurs at a rate that is lesser than what the rate of replication would have been had a subject not received a compound or formulation herein. In some embodiments, monitoring of telomere depletion in stem cells can determine that the depletion occurs at a rate that is lesser than what the rate would have been had a subject not received a compound or formulation herein. In some embodiments, quantitative evaluation of telomere length can determine that telomere length is longer than the telomere length would have been had a subject not received a compound or formulation herein. Cell division in a stem cell, replication of stem cells, and telomere depletion can be monitored in a stem cell obtained from a subject. Telomere length can be quantitatively evaluated in a stem cell obtained from a subject. In some embodiments, the stem cell can be obtained from a subject after the administration of a compound or formulation provided herein. In some embodiments, the stem cell can be obtained from a subject at least 1 month, 2 months, 3 months, 4 months, 5 months, 6 months, 7 months, 8 months, 9 months, 10 months, 11 months, 1 year, 2 years, 3 years, 4 years, or 5 years after administration of a compound or formulation provided herein. In some embodiments, the stem cell can be obtained from a subject at least 6 months after administration of a compound or formulation provided herein.

In some embodiments, the disclosure provides a method comprising administering to a subject an effective amount of a compound or formulation herein. In some embodiments, the compound or formulation herein can be administered in additional administrations to a subject based on the anti-aging effects or the reduction of deleterious effects of aging. In some embodiments, the compound or formulation herein can be administered in additional administrations to a subject based on the results of blocking pathways that promote unnecessary replication of stem cells. In some embodiments, the compound or formulation herein can be administered in additional administrations to a subject based at least in part on telomere length. In some embodiments, the compound or formulation herein can be administered in additional administrations to a subject based at least in part on monitoring of cell division in stem cells. In some embodiments, the compound or formulation herein can be administered in additional administrations to a subject based at least in part on monitoring of telomere depletion in a stem cell. In some embodiments, the compound or formulation herein can be administered in additional administrations to a subject based at least in part on monitoring of stem cell replication.

In some embodiments, chlorite salt or the chlorite ion thereof provides the anti-aging effect or reduces deleterious effects of aging preferentially, selectively, or exclusively in an inflammatory environment. In some embodiments, chlorite ion provides the anti-aging effect or reduces deleterious effects of aging exclusively in an inflammatory environment. In some embodiments, the inflammatory environment activates chlorite ion to form taurine chloramine, which exerts the anti-aging effect or reduces deleterious effect of aging on stem cells.

In some embodiments, chlorite salt or the chlorite ion thereof provides the anti-aging effect or reduces deleterious effects of aging by blocking unnecessary replication of stem cells, thereby slowing the depletion of telomeres in the stem cells, and thereby keeping the stem cells active. In some embodiments, chlorite salt or the chlorite ion thereof provides the anti-aging effect or reduces deleterious effects of aging by blocking unnecessary replication of stem cells, thereby slowing the depletion of telomeres in the stem cells, and thereby keeping the stem cells active, wherein the chlorite ion or salt blocks the unnecessary replication of stem cells in an inflammatory environment, wherein the inflammatory environment activates chlorite ion to form taurine chloramine.

In some embodiments, the disclosure provides a method comprising administering to a subject an effective amount of a chlorite salt, wherein the subject has a combined level of taurine and taurine chloramine in circulation that is deficient, and performing an assay on the subject, thereby determining that the administering the chlorite salt to the subject increases the combined level of taurine and taurine chloramine in circulation to a level that is not deficient. In some embodiments, the combined level of taurine and taurine chloramine in circulation that is deficient is no greater than any level provided herein. In some embodiments, the level that is not deficient is at least any level provided herein. In some embodiments, the subject was previously determined to suffer from a taurine deficiency as determined by a laboratory assay. In some embodiments, the subject is at least 40 years of age, or any age provided herein. In some embodiments, the method further comprises recommending endurance exercise to the subject. In some embodiments, the chlorite salt is sodium chlorite. In some embodiments, the effects of the administration persist at least a month after the administration, for example a level of TGFB1 in the subject remains elevated in the subject for at least one month after administration.

### Compounds of the Disclosure

Disclosed herein are oxidative agents that can treat a condition with immune system dysregulation. In some embodiments, an oxidative agent of the disclosure can treat a condition with immune system dysregulation by inactivating macrophages. In some embodiments, the compound of the disclosure can be rapidly converted into a regulator of NFkB activation.

In some embodiments, the compound of the disclosure is chlorite (ClO₂⁻) or a pharmaceutically-acceptable salt thereof. In some embodiments, the compound of the disclosure is sodium chlorite (NaClO₂). In some embodiments, the compound of the disclosure is potassium chlorite (KClO₂). In some embodiments, sodium chlorite can be converted from a prodrug to an intracellular form of taurine chloramine (TauCl) via a hyperchlorite intermediate. TauCl is a long-lived effector molecule within macrophages that down-regulates NF-kB expression and inhibits production of pro-inflammatory cytokines in part through activation of heme oxygenase-1 (HO-1).

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure. In some embodiments, a compound of the disclosure can be at least about 90% pure. In some embodiments, a compound of the disclosure can be at least about 95% pure. In some embodiments, a compound of the disclosure can be at least about 98% pure. In some embodiments, a compound of the disclosure can be at least about 99% pure. In some embodiments, a compound of the disclosure can be at least about 99.5% pure. In some embodiments, a compound of the disclosure can be at least about 99.8% pure.

### Chlorite Compositions

In some embodiments, the sodium chlorite described herein is an aqueous pharmaceutical formulation. In some embodiments, the aqueous pharmaceutical formulation comprises sodium chlorite and a saline solution.

In some embodiments, the pH of the aqueous pharmaceutical formulation is about 7 to about 11. In some embodiments, the pH of the aqueous pharmaceutical formulation is about 7 to about 9.5. In some embodiments, the aqueous pharmaceutical formulation includes a buffer. Non-limiting examples of buffers include phosphate buffers, borate buffers, citrate buffers, and carbonate buffers. In some embodiments, the buffer is a phosphate buffer such as monosodium phosphate or disodium phosphate. In some embodiments, any of the formulations or pharmaceutical formulations described herein comprises a pH adjusting agent that consists essentially of a phosphate salt or a mixture of phosphate salts.

In some embodiments, the pH of a chlorite formulation for use with the present disclosure can be adjusted to between about 7 and about 11.5. In some embodiments, the pH of a chlorite formulation is lowered to between about 7 and about 11.5 using a pH adjusting compound that does not expose the formulation to high local acidity. In some embodiments, the pH adjusting compound is any one or more of monosodium phosphate, disodium phosphate, or acetic acid.

In some embodiments, the pH adjusting agent(s) or pH adjusting compound(s) are weak acids or weak bases having a pKa of about 4 to about 9, a pKa of about 5 to about 9, or a pKa of about 5 to about 8, or a pKa of about 6 to about 7.5. Examples include, but are not limited to, a phosphate buffer having a pKa of about 4 to about 9, for example, monobasic phosphates, or monosodium phosphate and/or disodium phosphate and lower alkanoic acids, for example, acetic acid or propionic acid. In some embodiments, the pH of a formulation sensitive to acidity is lowered to between about 7 and about 11.5 using a pH adjusting compound that does not expose the formulation to acidity, including but not limited to a high local acidity in the area around the pH adjusting compound. In some embodiments, the pH of a formulation sensitive to acidity is lowered to between about 7 and about 10 using a pH adjusting compound that does not expose the formulation to acidity, including but not limited to a high local acidity in the area around the pH adjusting compound. In some embodiments, the pH of a formulation sensitive to acidity is lowered to between about 7 and about 9.5 using a pH adjusting compound that does not expose the formulation to acidity, including but not limited to a high local acidity in the area around the pH adjusting compound. In some embodiments, the pH of a formulation sensitive to acidity is lowered to between about 7 and about 9.0 using a pH adjusting compound that does not expose the formulation to acidity, including but not limited to a high local acidity in the area around the pH adjusting compound. In some embodiments, the pH of a formulation sensitive to acidity is lowered to between about 7 and about 8.5 using a pH adjusting compound that does not expose the formulation to acidity, including but not limited to a high local acidity in the area around the pH adjusting compound. In some embodiments, the pH of a formulation sensitive to acidity is lowered to between about 7.1 and about 7.7 using a pH adjusting compound that does not expose the formulation to acidity, including but not limited to a high local acidity in the area around the pH adjusting compound.

Injectable preparations, for example, sterile injectable aqueous or oleaginous suspensions can be formulated, for example, using suitable dispersing or wetting agents and suspending agents. An injectable preparation can be a sterile, injectable solution, suspension, or emulsion in a nontoxic, parenterally-acceptable diluent or solvent, for example, as a solution in 1,3-butanediol. Non-limiting examples of suitable vehicles and solvents include Water for Injection (WFI, USP), Sterile Water for Injection (SWFI, USP), Ringer's solution, USP, and isotonic solution, such as isotonic sodium chloride solution. In some embodiments, a sterile, fixed oil is used as a solvent or suspending medium. In some embodiments, a fatty acid such as oleic acid is formulated in an injectable.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use. In some embodiments, the injectable formulations are sterile, pyrogen free, and free of particulates according to USP-NF standards. In some embodiments, sterility, pyrogenicity, and particulates assays are conducted according to USP-NF protocols.

In some embodiments, the pharmaceutically acceptable chlorite salt is administered in an amount ranging from 0.1 to 10 mg/kg body mass. In some embodiments, the chlorite salt is administered more than once in a month, such as at least once per week for a period of at least one month. In some embodiments, the chlorite salt is administered for at least a year.

In some embodiments, the chlorite formulations for use with the present invention comprise low amounts of chlorate ion, sulfate ion, or chloride ion. In some embodiments, the composition is substantially free of sulfate ion.

In some embodiments, a formulation herein contains an amount of chloride ions than is less than about 1.9% of the chlorite ions by mass in the formulation, for example, less than about 1.8%; less than about 1.5%; less than about 1.0%; less than about 0.5%; less than about 0.3%; less than about 0.1%; less than about 0.05%; less than about 0.01%; less than about 0.001%; from about 0.001 to about 0.1%; from about 0.1 to about 0.5%; from about 0.5 to about 1.0%; from about 1.0 to about 1.5%; or from about 1.5 to about 1.8%.

In some embodiments, a formulation herein contains less than an amount of chlorate ions that is less than about 1.5% of chlorate ions by mass in the formulation, for example, less than about 1.4%, less than about 1.3%; less than about 1.0%; less than about 0.5%; less than about 0.3%; less than about 0.10%; less than about 0.010%; less than about 0.0010%; from about 0.001 to about 0.1%; from about 0.001 to about 0.01%; from about 0.01 to about 0.1%; from about 0.1 to about 0.5%; from about 0.5 to about 1.0%; or from about 1.0 to about 1.4% of chlorate ions. In some embodiments, a formulation herein is substantially free of chlorate ions. In some embodiments, a formulation herein contains less than about 0.5% of chlorate ions by mass in the formulation. In some embodiments, a formulation herein contains less than about 0.19% of chlorate ions by mass in the formulation. In some embodiments, a formulation herein contains less than about 0.1% of chlorate ions by mass in the formulation. In some embodiments, the level of chlorate ions is below the level of detection using HPLC.

In some embodiments, a formulation herein contains less than an amount of sulfate ions that is less than about 0.7% of sulfate ions by mass in the formulation, for example, less than about 0.65%, less than about 0.6%; less than about 0.5%; less than about 0.4%; less than about 0.3%; less than about 0.2%; less than about 0.1%; less than about 0.08%; less than about 0.07%; less than about 0.06%; less than about 0.05%; less than about 0.005%; less than about 0.0005%; from about 0.001 to about 0.1%; from about 0.01 to about 1%; from about 0.01 to about 0.5%; from about 0.06 to about 0.08%; or from about 0.5 to about 0.65%. In some embodiments, a formulation herein is substantially free of sulfate ions. In some embodiments, a formulation herein contains less than about 0.5% of sulfate ions by mass in the formulation. In some embodiments, a formulation herein contains less than about 0.08% of sulfate ions by mass in the formulation. In some embodiments, the level of sulfate ions is below the level of detection using HPLC.

In some embodiments, a formulation herein contains less than an amount of chloride ions that is less than about 0.5% of chloride ions by mass in the formulation, for example, less than about 0.24%, less than about 0.2%; or less than about 0.1%. In some embodiments, a formulation herein contains less than about 0.2% of chloride ions by mass in the formulation. In some embodiments, a formulation herein contains less than about 0.1% of chloride ions by mass in the formulation. In some embodiments, the level of chloride ions is below the level of detection using HPLC.

In some embodiments, the chlorite formulations described herein comprise no greater than about 10% by weight of by products or impurities present in commercially available technical grade chlorite. Non-limiting examples of by-products or impurities present in commercially available technical grade chlorite include chlorate, sulfate, chlorine dioxide, chloride, sodium bicarbonate, and sodium carbonate. In some embodiments, the chlorite formulations described herein comprise no greater than about any of 15%, about 12%, about 10%, about 9%, about 8%, about 7%, about 6%, about 5%, about 4%, about 3%, about 2%, about 1%, about 0.5%, about 0.3%, about 0.1%, between about 0.1 to about 5%; between about 5 to about 10%; or between about 10 to about 15% by weight of one or more degradation products or impurities present in commercially available technical grade sodium chlorite. In some embodiments, the chlorite formulations described herein comprise no greater than about 0.5% by weight of degradation products or impurities present in commercially available technical grade sodium chlorite. In some embodiments, the chlorite formulations described herein comprise no greater than about 5% by weight of degradation products or impurities present in commercially available technical grade sodium chlorite. In some embodiments, the sodium chlorite formulations described herein are substantially free of the degradation products or impurities present in commercially available technical grade sodium chlorite.

In some embodiments, the chlorite formulations described herein comprise chlorite salt at a concentration of about 1 millimolar (mM), about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 70 mM, about 80 mM, about 90 mM, or about 100 mM. In some embodiments, the chlorite formulations described herein comprises chlorite salt at a concentration of about 62 mM.

In some embodiments, the chlorite formulations described herein comprise purified sodium chlorite. In some embodiments, the chlorite formulations described herein comprise purified sodium chlorite, wherein the purified sodium chlorite comprises no more than 2.0% sodium chloride, wherein the purified sodium chlorite comprises no more than 1.0% sodium chlorate, and wherein the chlorite composition comprises dibasic sodium phosphate and has a pH between 7.0 and 9.5.

In some embodiments, the chlorite formulations described herein comprise purified sodium chlorite, wherein the purified sodium chlorite comprises no more than about 1.0% sodium chloride, wherein the purified sodium chlorite comprises no more than about 1.0% sodium chlorate, and wherein the chlorite composition comprises a buffer and has a pH between about 7 and about 8. In some embodiments, the chlorite formulations described herein comprise purified sodium chlorite, wherein the purified sodium chlorite comprises no more than about 0.8% sodium chloride, wherein the purified sodium chlorite comprises no more than about 0.8% sodium chlorate, and wherein the chlorite composition comprises a buffer and has a pH between about 7 and about 8. In some embodiments, the chlorite formulations described herein comprise purified sodium chlorite, wherein the purified sodium chlorite comprises no more than about 0.6% sodium chloride, wherein the purified sodium chlorite comprises no more than about 0.6% sodium chlorate, and wherein the chlorite composition comprises a buffer and has a pH between about 7 and about 8. In some embodiments, the chlorite formulations described herein comprise purified sodium chlorite, wherein the purified sodium chlorite comprises no more than about 0.5% sodium chloride, wherein the purified sodium chlorite comprises no more than about 0.5% sodium chlorate, and wherein the chlorite composition comprises a buffer and has a pH between about 7 and about 8. In some embodiments, the chlorite formulations described herein comprise purified sodium chlorite, wherein the purified sodium chlorite comprises no more than about 0.5% sodium chloride, wherein the purified sodium chlorite comprises no more than about 0.5% sodium chlorate, and wherein the chlorite composition comprises a buffer and has a pH between about 7.5 and about 7.7.

In some embodiments, the chlorite formulations described herein can have a volume of about 10 milliliter (mL), about 20 mL, about 30 mL, about 40 mL, about 50 mL, about 60 mL, about 70 mL, about 80 mL, about 90 mL, about 100 mL, about 110 mL, about 120 mL, about 130 mL, about 140 mL, about 150 mL, about 160 mL, about 170 mL, about 180 mL, about 190 mL, about 200 mL, about 210 mL, about 220 mL, about 230 mL, about 240 mL, about 250 mL, about 260 mL, about 270 mL, about 280 mL, about 290 mL, about 300 mL, about 350 mL, about 400 mL, about 450 mL, about 500 mL, or about 1000 mL. In some embodiments, the chlorite formulations described herein can have a volume of about 250 mL.

The regimen of administration can involve administration in an amount and at a frequency to provide a desired effect. For example, chlorite or a chlorite-containing agent can be administered for 2, 3, 4, 5, 6, 7, 8, 9, 10, or more consecutive days. Administration can be daily, for example, once a day. In some embodiments, sodium chlorite is administered daily. Administration can be every other day. In some embodiments, sodium chlorite is administered every other day. Administration can be intermittent, for example 3 days per month. In some embodiments, sodium chlorite is administered intermittently.

In some embodiments, the pharmaceutical composition can be administered in a cycle. A non-limiting example of a cycle provides: a) a first period of time wherein the pharmaceutical composition is administered at a first dose for a first number of times; and b) a second period of time wherein the pharmaceutical composition is administered at a second dose for a second number of times. In some embodiments, the cycle is performed about 2-4 times.

In some embodiments, the dosing schedule provides periods of administration alternating with periods of non-administration. In some embodiments, sodium chlorite is administered in a four-week cycle. In some embodiments, sodium chlorite is administered in a three-week cycle. In some embodiments, the cycle could be repeated as necessary to achieve the desired result. In some embodiments, sodium chlorite is administered in a two-week cycle. In some embodiments, a total of 2-4 cycles are performed. In some embodiments, a total of about 4 cycles, about 5 cycles, about 6 cycles, about 7 cycles, about 8 cycles, about 9 cycles, about 10 cycles, about 11 cycles, about 12 cycles, about 13 cycles, or about 14 cycles are performed. In some embodiments, a total of about 6 cycles are performed. In some embodiments, a total of about 10 cycles are performed. In some embodiments, a total of about 12 cycles are performed.

In some embodiments, the chlorite formulation described herein is administered daily for a first number of days in a row in first month and administered daily for a second number of days in subsequent months. In some embodiments, the first number of days is about 2, about 3, about 4, about 5, about 6, or about 7. In some embodiments, the first number of days is about 2. In some embodiments, the first number of days is about 3. In some embodiments, the first number of days is about 4. In some embodiments, the first number of days is about 5. In some embodiments, the first number of days is about 6. In some embodiments, the first number of days is about 7. In some embodiments, the first number of days is about 2. In some embodiments, the second number of days is about 2. In some embodiments, the second number of days is about 3. In some embodiments, the second number of days is about 4. In some embodiments, the second number of days is about 5. In some embodiments, the second number of days is about 6. In some embodiments, the second number of days is about 7.

In some embodiments, the chlorite formulation described herein is initially administered daily for 5 days in a row in the first month, and daily for 3 days in a row in subsequent months. In some embodiments, the chlorite formulation described herein is initially administered daily for 5 days in a row in the first month, and daily for 3 days in a row in subsequent months, wherein the chlorite formulation comprises purified sodium chlorite, wherein the purified sodium chlorite is at least 97% pure, wherein the purified sodium chlorite comprises no more than 2.0% sodium chloride, wherein the purified sodium chlorite comprises no more than 1.0% sodium chlorate, and wherein the chlorite composition comprises dibasic sodium phosphate and has a pH between about 7.5 and about 9.5. In some embodiments, the chlorite formulation described herein is initially administered daily for 5 days in a row in the first month, and daily for 3 days in a row in subsequent months, wherein the chlorite formulation comprises purified sodium chlorite, wherein the purified sodium chlorite is at least 97% pure, wherein the purified sodium chlorite comprises no more than 2.0% sodium chloride, wherein the purified sodium chlorite comprises no more than 1.0% sodium chlorate, and wherein the chlorite composition comprises dibasic sodium phosphate and has a pH between about 7.5 and about 8.5. In some embodiments, the chlorite formulation described herein is initially administered daily for 5 days in a row in the first month, and daily for 3 days in a row in subsequent months, wherein the chlorite formulation comprises purified sodium chlorite, wherein the purified sodium chlorite is at least 97% pure, wherein the purified sodium chlorite comprises no more than 2.0% sodium chloride, wherein the purified sodium chlorite comprises no more than 1.0% sodium chlorate, and wherein the chlorite composition comprises dibasic sodium phosphate and has a pH between about 7.5 and about 8.

Any compositions and pharmaceutical formulation described herein can be used in kits. In some embodiments, the kits are intended for administration of sodium chlorite or a sodium chlorite-containing agent, or pharmaceutical formulations comprising such agents. The kits can include a unit dosage amount of the agents or formulations as described herein. In some variations, the kits comprise suitable packaging. In some embodiments, the kits comprise instructions for use of the active agent in a neurodegenerative disease as described above. In some embodiments, the kit contains instructions for using sodium chlorite formulations to treat a neurodegenerative disease as described herein. In some embodiments, a kit contain suitable instructions for practicing any of the treatment methods described herein with a formulation described herein. In some embodiments, a kit is used to treat any one or more of the diseases or conditions described herein. A kit can comprise an aid to administration of the active agent formulation, such as a syringe for injection or pressure pack for oral forms.

### Pharmaceutical Compositions

A pharmaceutical composition of the invention can be a combination of any compounds described herein with other chemical components, such as carriers, stabilizers, solubilizers, tonicity enhancing agents, buffers, preservatives, diluents, dispersing agents, suspending agents, thickening agents, and/or excipients. The pharmaceutical compositions can include at least one pharmaceutically-acceptable carrier, stabilizer, solubilizer, tonicity enhancing agent, buffer, preservative, diluent, dispersing agent, suspending agent, thickening agent, and/or excipients and a compound described herein as free-base or pharmaceutically-acceptable salt form. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration. Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

In some embodiments, a pharmaceutical composition can be formulated for oral administration. In some embodiments, a pharmaceutical composition formulated for oral administration can be formulated by combining one or more compounds of the disclosure with one or more pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

In some embodiments, a pharmaceutical composition can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use.

In some embodiments, a pharmaceutical composition can be formulated for topical administration. In some embodiments, a pharmaceutical composition can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives. In some embodiments, a pharmaceutical composition of the disclosure can be applied topically to the skin, or a body cavity, for example, oral, vaginal, bladder, cranial, spinal, thoracic, or pelvic cavity of a subject. In some embodiments, a compound or pharmaceutical composition of the disclosure can be applied to an accessible body cavity.

In practicing the methods of treatment or use provided herein, therapeutically-effective amounts of the compounds described herein are administered in pharmaceutical compositions to a subject having a disease or condition to be treated. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions comprising a compound described herein can be manufactured, for example, by mixing, dissolving, emulsifying, encapsulating, entrapping, or compression processes. Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, elixir, nanosuspension, aqueous or oily suspensions, drops, syrups, and any combination thereof.

Non-limiting examples of pharmaceutically-acceptable excipients suitable for use include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, granulating agents, lubricating agents, sweetening agents, glidants, gums, flavoring agents, plant cellulosic material, and any combination thereof.

A pharmaceutical composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release formulation. A delayed release formulation can be formulated to delay a compound's action for an extended period of time. A delayed release form can be formulated to delay the release of an effective dose of one or more compounds, for example, for about 4, about 8, about 12, about 16, or about 24 hours.

In some embodiments, a controlled release formulation can be a sustained release formulation. A sustained release formulation can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), each of which is incorporated by reference in its entirety.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

A pharmaceutical composition can have a pH adjusted to from about 7 and to about 11.5. In some embodiments, the pH of a pharmaceutical composition of the disclosure can be adjusted using an agent that does not expose the formulation to high local acidity. In some embodiments, the pH of a pharmaceutical composition of the disclosure can be adjusted using, for example, monosodium phosphate, disodium phosphate, dibasic sodium phosphate, or acetic acid.

In some embodiments, a pharmaceutical composition of the disclosure can have a pH of from about 7 to about 7.5, from about 7.5 to about 8, from about 8 to about 8.5, from about 8.5 to about 9, from about 9 to about 9.5, from about 9.5 to about 10, from about 10 to about 10.5, from about 10.5 to about 11, from about 11 to about 11.5, or from about 11.5 to about 12. In some embodiments, a pharmaceutical composition of the disclosure can have a pH of from about 7 to about 7.5. In some embodiments, a pharmaceutical composition of the disclosure can have a pH of from about 7.5 to about 8. In some embodiments, a pharmaceutical composition of the disclosure can have a pH of from about 8 to about 8.5. In some embodiments, a pharmaceutical composition of the disclosure can have a pH of from about 7.5 to about 9.5.

In some embodiments, a pharmaceutical composition of the disclosure can have a pH of less than about 12. In some embodiments, a pharmaceutical composition of the disclosure can have a pH of less than about 11.5, less than about 11, less than about 10.5, less than about 10, less than about 9.5, less than about 9, less than about 8.5, less than about 8, less than about 7.5, less than about 7, less than about 6.5, or less than about 6. In some embodiments, the pH of a pharmaceutical composition of the disclosure can have a pH of less than about 11.5. In some embodiments, a pharmaceutical composition of the disclosure can have a pH of less than about 10.5. In some embodiments, a pharmaceutical composition of the disclosure can have a pH of less than about 8.5. In some embodiments, a pharmaceutical composition of the disclosure can have a pH of less than about 7.5. In some embodiments, a pharmaceutical composition of the disclosure has a pH of about 7.4. In some embodiments, a pharmaceutical composition of the disclosure has a pH that is at a physiological level.

In some embodiments, a pharmaceutical composition of the disclosure can comprise a solvent. In some embodiments, a pharmaceutical composition of the disclosure can comprise water as a solvent. In some embodiments, a pharmaceutical composition of the disclosure is at least about 50%, at least about 55%, at least about 60%, at least about 65%, at least about 70%, at least about 75%, at least about 80%, at least about 85%, at least about 90%, or at least about 95% (v/v) water. In some embodiments, a pharmaceutical composition of the disclosure is at least about 80% (v/v) water. In some embodiments, a pharmaceutical composition of the disclosure is at least about a 90% (v/v) water. In some embodiments, a pharmaceutical composition of the disclosure is at least about a 95% (v/v) water. In some embodiments, a pharmaceutical composition of the disclosure is at least about a 98% (v/v) water.

In some embodiments, a pharmaceutical composition of the disclosure is from about 50% to about 60%, from about 60% to about 70%, from about 70% to about 80%, from about 80% to about 90%, or from about 90% to about 98% (v/v) water. In some embodiments, a pharmaceutical composition of the disclosure is from about 80% to about 90% (v/v) water. In some embodiments, a pharmaceutical composition of the disclosure is from about 90% to about 95% (v/v) water. In some embodiments, a pharmaceutical composition of the disclosure is from about 90% to about 98% (v/v) water.

In some embodiments, a pharmaceutical composition comprises from about 1 μM to about 1.5 M of a compound of the disclosure. In some embodiments, a pharmaceutical composition comprises from about 1 μM to about 10 μM, from about 10 μM to about 50 μM, from about 50 μM to about 100 μM, from about 0.1 mM to about 0.5 mM, from about 0.5 mM to about 1 mM, from about 1 mM to about 25 mM, from about 25 mM to about 100 mM, from about 100 mM to about 250 mM, from about 250 mM to about 500 mM, from about 500 mM to about 750 mM, or from about 750 mM to about 1000 mM of a compound of the disclosure. In some embodiments, a pharmaceutical composition comprises from about 1 μM to about 10 μM of a compound of the disclosure. In some embodiments, a pharmaceutical composition comprises from about 10 μM to about 50 μM of a compound of the disclosure. In some embodiments, a pharmaceutical composition comprises from about 50 μM to about 100 μM of a compound of the disclosure. In some embodiments, a pharmaceutical composition comprises from about 100 μM to about 250 μM of a compound of the disclosure. In some embodiments, a pharmaceutical composition comprises from about 250 μM to about 500 μM of a compound of the disclosure. In some embodiments, a pharmaceutical composition comprises from about 1 mM to about 5 mM of a compound of the disclosure.

In some embodiments, a pharmaceutical composition comprises at least about 1 μM of a compound of the disclosure. In some embodiments, a pharmaceutical composition comprises at least about 1 μM, at least about 10 μM, at least about 50 μM, at least about 100 μM, at least about 250 μM, at least about 500 μM, at least about 750 μM, at least about 1 mM, at least about 25 mM, at least about 50 mM, at least about 75 mM, at least about 100 mM, at least about 250 mM, at least about 500 mM, at least about 750 mM, or at least about 1000 mM of a compound of the disclosure. In some embodiments, a pharmaceutical composition comprises at least about 1 μM of a compound of the disclosure. In some embodiments, a pharmaceutical composition comprises at least about 10 μM of a compound of the disclosure. In some embodiments, a pharmaceutical composition comprises at least about 25 μM of a compound of the disclosure. In some embodiments, a pharmaceutical composition comprises at least about 50 μM of a compound of the disclosure. In some embodiments, a pharmaceutical composition comprises at least about 60 μM of a compound of the disclosure. In some embodiments, a pharmaceutical composition comprises at least about 65 μM of a compound of the disclosure. In some embodiments, a pharmaceutical composition comprises at least about 100 μM of a compound of the disclosure. In some embodiments, a pharmaceutical composition comprises at least about 250 μM of a compound of the disclosure. In some embodiments, a pharmaceutical composition comprises at least about 500 μM of a compound of the disclosure.

In some embodiments, a pharmaceutical composition comprises about 1 μM of a compound of the disclosure. In some embodiments, a pharmaceutical composition comprises about 1 μM, about 10 μM, about 50 μM, about 100 μM, about 250 μM, about 500 μM, about 750 μM, about 1 mM, about 25 mM, about 50 mM, about 75 mM, about 100 mM, about 250 mM, about 500 mM, about 750 mM, or about 1000 mM of a compound of the disclosure. In some embodiments, a pharmaceutical composition comprises about 1 μM of a compound of the disclosure. In some embodiments, a pharmaceutical composition comprises about 10 μM of a compound of the disclosure. In some embodiments, a pharmaceutical composition comprises about 25 μM of a compound of the disclosure. In some embodiments, a pharmaceutical composition comprises about 50 μM of a compound of the disclosure. In some embodiments, a pharmaceutical composition comprises about 100 μM of a compound of the disclosure. In some embodiments, a pharmaceutical composition comprises about 250 μM of a compound of the disclosure. In some embodiments, a pharmaceutical composition comprises about 500 μM of a compound of the disclosure.

In some embodiments, a pharmaceutical composition of the disclosure comprises at least about 0.5 mg/mL of a compound of the disclosure. In some embodiments, a pharmaceutical composition of the disclosure comprises at least about 0.5 mg/mL, at least about 1 mg/mL, at least about 1.5 mg/mL, at least about 2 mg/mL, at least about 2.5 mg/mL, at least about 3 mg/mL, at least about 3.5 mg/mL, at least about 4 mg/mL, at least about 4.5 mg/mL, at least about 5 mg/mL, at least about 5.5 mg/mL, at least about 6 mg/mL, at least about 6.5 mg/mL, at least about 7 mg/mL, at least about 7.5 mg/mL, at least about 8 mg/mL, at least about 8.5 mg/mL, at least about 9 mg/mL, at least about 9.5 mg/mL, or at least about 10 mg/mL of a compound of the disclosure. In some embodiments, a pharmaceutical composition of the disclosure comprises at least about 2 mg/mL of a compound of the disclosure. In some embodiments, a pharmaceutical composition of the disclosure comprises at least about 4 mg/mL of a compound of the disclosure. In some embodiments, a pharmaceutical composition of the disclosure comprises at least about 5 mg/mL of a compound of the disclosure. In some embodiments, a pharmaceutical composition of the disclosure comprises at least about 5.6 mg/mL of a compound of the disclosure. In some embodiments, a pharmaceutical composition of the disclosure comprises at least about 5.5 mg/mL of sodium chlorite. In some embodiments, a pharmaceutical composition of the disclosure comprises at least about 4 mg/mL of chlorite.

In some embodiments, a pharmaceutical composition of the disclosure comprises from about 0.5 mg/mL to about 10 mg/mL of a compound of the disclosure. In some embodiments, a pharmaceutical composition of the disclosure comprises from about 0.5 mg/mL to about 1 mg/mL, from about 1 mg/mL to about 2 mg/mL, from about 2 mg/mL to about 3 mg/mL, from about 3 mg/mL to about 4 mg/mL, from about 4 mg/mL to about 5 mg/mL, from about 5 mg/mL to about 6 mg/mL, from about 6 mg/mL to about 7 mg/mL, from about 7 mg/mL to about 8 mg/mL, from about 8 mg/mL to about 9 mg/mL, or from about 9 mg/mL to about 10 mg/mL of a compound of the disclosure. In some embodiments, a pharmaceutical composition of the disclosure comprises from about 4 mg/mL to about 5 mg/mL of a compound of the disclosure. In some embodiments, a pharmaceutical composition of the disclosure comprises from about 5 mg/mL to about 6 mg/mL of a compound of the disclosure. In some embodiments, a pharmaceutical composition of the disclosure comprises from about 4 mg/mL to about 5 mg/mL of chlorite. In some embodiments, a pharmaceutical composition of the disclosure comprises from about 5 mg/mL to about 6 mg/mL of sodium chlorite.

In some embodiments, a pharmaceutical composition of the disclosure comprises about 0.5 mg/mL of a compound of the disclosure. In some embodiments, a pharmaceutical composition of the disclosure comprises about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, or about 10 mg/mL of a compound of the disclosure. In some embodiments, a pharmaceutical composition of the disclosure comprises about 2 mg/mL of a compound of the disclosure. In some embodiments, a pharmaceutical composition of the disclosure comprises about 4 mg/mL of a compound of the disclosure. In some embodiments, a pharmaceutical composition of the disclosure comprises about 5 mg/mL of a compound of the disclosure. In some embodiments, a pharmaceutical composition of the disclosure comprises about 5.6 mg/mL of a compound of the disclosure. In some embodiments, a pharmaceutical composition of the disclosure comprises about 5.5 mg/mL of sodium chlorite.

In some embodiments, a pharmaceutical composition of the disclosure comprises about 4 mg/mL of chlorite.

In some embodiments, a pharmaceutical composition of the disclosure is formulated for intravenous administration. In some embodiments, a pharmaceutical composition of the disclosure comprises dibasic sodium phosphate. In some embodiments, a pharmaceutical composition of the disclosure has a pH of from about 7.0 to about 9.5. In some embodiments, a pharmaceutical composition of the disclosure has a pH of from about 7.5 to about 9.0 In some embodiments, the pharmaceutical composition of the disclosure does not comprise any preservatives. In some embodiments, the pharmaceutical composition of the disclosure comprises about 4 mg/mL of chlorite. In some embodiments, the pharmaceutical composition of the disclosure comprises about 5.5 mg/mL of sodium chlorite.

### Methods of Use

In some embodiments, a compound or formulation provided herein can reduce deleterious effects of aging. In some embodiments, a compound or formulation provided herein can reduce deleterious effects of aging associated with over-replication of stem cells.

In some embodiments, a compound or formulation provided herein can slow progression of a disease. In some embodiments, a compound or formulation provided herein can halt progression of a disease. In some embodiments, a compound or formulation provided herein can modulate levels of taurine chloramine in a subject. In some embodiments, a compound or formulation provided herein can increase levels of taurine chloramine in a subject. In some embodiments, a compound or formulation provided herein can stabilize levels of taurine chloramine in a subject.

In some embodiments, a compound or formulation provided herein can restore innate immune function in a subject. In some embodiments, a compound or formulation provided herein can modulate levels of a biomarker associated with activation of the innate immune system. In some embodiments, a compound or formulation provided herein can modulate levels of a biomarker associated with activation of the innate immune system in a subject with high levels of CRP (e.g., CRP >1.13 mg/L). In some embodiments, a compound or formulation provided herein can modulate levels of serum amyloid A (SAA) in a subject. In some embodiments, a compound or formulation provided herein can modulate levels of serum amyloid A (SAA) in a subject with high levels of CRP. In some embodiments, a compound or formulation provided herein can decrease levels of SAA. In some embodiments, a compound or formulation provided herein can modulate levels of plasma TGFB1 in a subject. In some embodiments, a compound or formulation provided herein can modulate levels of plasma TGFB1 in a subject with high levels of CRP. In some embodiments, a compound or formulation provided herein can modulate the activity of the innate immune alpha 2 macroglobulin (A2M) system. In some embodiments, a compound or formulation provided herein can modulate the activity of the A2M system in a subject with high levels of CRP.

In some embodiments, a compound or formulation provided herein can modulate the innate immune response in a subject. In some embodiments, a compound or formulation provided herein can modulate the innate immune response mediated by LPS in a subject. In some embodiments, a compound or formulation provided herein can modulate the expression of genes involved in the innate immune response in a subject. In some embodiments, a compound or formulation provided herein can modulate the activity of genes involved in the innate immune response in a subject. In some embodiments, a compound or formulation provided herein can modulate the activation of genes involved in the innate immune response in a subject. In some embodiments, a compound or formulation provided herein can modulate the levels of genes involved in the innate immune response in a subject. In some embodiments, a compound or formulation provided herein can decrease the activation of the innate immune response in a subject. In some embodiments, a compound or formulation provided herein can reverse the activation of the innate immune response in a subject. In some embodiments, a compound or formulation provided herein can modulate macrophage activation in a subject. In some embodiments, a compound or formulation provided herein can decrease macrophage activation in a subject. In some embodiments, a compound or formulation provided herein can reverse macrophage activation in a subject. In some embodiments, a compound or formulation provided herein can modulate levels of stimuli associated with macrophage activation. In some embodiments, a compound or formulation provided herein can decrease levels of stimuli associated with macrophage activation. In some embodiments, a compound or formulation provided herein can modulate macrophage dysfunction in a subject. In some embodiments, a compound or formulation provided herein can decrease macrophage dysfunction in a subject. In some embodiments, a compound or formulation provided herein can reverse macrophage dysfunction in a subject.

In some embodiments, a compound or formulation provided herein can regulate microbial translocation. During microbial translocation, microbes and microbial products translocate across the tight epithelial barrier into systemic circulation. Microbial translocation is a self-perpetuating proinflammatory process implicated in diseases, including ALS. In ALS, microbial translocation is hypothesized to proceed according to the following mechanism: (1) abnormal neurons stimulate local microglia to produce proinflammatory factors that activate blood monocytes to become proinflammatory; (2) inflammatory macrophages do not phagocytose microorganisms or participate in wound healing, resulting in leakage of bacterial and bacterial products in the blood; (3) bacterial endotoxins (e.g., LPS) activate blood monocytes and tissue macrophages, leading to a systemic proinflammatory state; (4) monocytes are recruited from the blood into damaged tissues to initiate repair; however, endotoxin activation results in trafficking of non-wound healing, non-phagocytic, proinflammatory cells into damaged tissues, leading to disease persistence. In some embodiments, a compound or formulation provided herein can modulate immunologic dysfunction driven by microbial translocation. In some embodiments, a compound or formulation provided herein can decrease immunological dysfunction driven by microbial translocation. In some embodiments, a compound or formulation provided herein can decrease immunological dysfunction driven by microbial translocation. In some embodiments, a compound or formulation provided herein can modulate immune activation associated with microbial translocation. In some embodiments, a compound or formulation provided herein can decrease immune activation associated with microbial translocation. In some embodiments, a compound or formulation provided herein can reverse immune activation associated with microbial translocation. In some embodiments, a compound or formulation provided herein can modulate endotoxin-driven inflammation-mediated repression of myeloid precursor function. In some embodiments, a compound or formulation provided herein can decrease endotoxin driven inflammation-mediated repression of myeloid precursor function. In some embodiments, a compound or formulation provided herein can reverse endotoxin-driven inflammation-mediated repression of myeloid precursor function. In some embodiments, a compound or formulation provided herein can regulate plasma biomarkers associated with microbial translocation. In some embodiments, a compound or formulation provided herein can increase biomarkers associated with microbial translocation. In some embodiments, a compound or formulation provided herein can decrease biomarkers associated with microbial translocation. In some embodiments, a compound or formulation provided herein can increase the expression, level, or activity of biomarkers including EGF (epidermal growth factor), IL-10 (interleukin 10), and neopterin. In some embodiments, a compound or formulation provided herein can decrease the expression, level, or activity of biomarkers including IL-18 (interleukin 18), LPS, LBP (lipopolysaccharide binding protein), soluble CD163 (sCD163), and HGF (hepatocyte growth factor). In some embodiments, a compound or formulation provided herein can downregulate NF-κB and proinflammatory factors. In some embodiments, a compound or formulation provided herein can increase expression, levels, or activity of EGF (epidermal growth factor). In some embodiments, a compound or formulation provided herein can convert macrophages to a phagocytic state. In some embodiments, a compound or formulation provided herein can decrease levels of sCD163.

In some embodiments, a compound or formulation provided herein can modulate signaling pathways in HSCs. In some embodiments, a compound or formulation provided herein can modulate signaling pathways in HSCs stimulated with LPS. Non-limiting examples of pathways that can be modulated by a compound or formulation provided herein include response to stress, response to external stimulus, immune system process, defense response, biological process involved in interspecies interaction between organism, immune response, response to biotic stimulus, response to other organism, response to external biotic stimulus, defense response to other organism, regulation of immune system process, innate immune response, response to cytokine, cellular response to cytokine stimulus, regulation of response to external stimulus, inflammatory response, regulation of immune response, cytokine production, response to virus, cytokine-mediated signaling pathway, defense response to virus, and defense response to symbiont.

In some embodiments, a compound or formulation provided herein can modulate pathways related to “defense response to other organism” in HSCs. Non-limiting examples of gene products involved in “defense response to other organism” in HSCs include APOBEC3A, AXL, BATF2, CCL13, CCL7, CCL8, CD207, CFB, CMPK2, CXCL10, DDX60, DDX60L, DHX58, EIF2AK2, EPHB2, GBP1, HERC5, HERC6, IFI27, IF1I27, IF44, IF44L, 1F16, IFIH1, IFIT1, IFIT2, IFIT3, IFIT5, IRF7, ISG15, LEAP2, LYG1, MX1, MX2, OAS1, OAS2, OAS3, OASL, PARP9, RSAD2, STAT1, UBD, USP18, and ZBP1. In some embodiments, a compound or formulation provided herein can modulate gene and gene products related to CCL13. CCL13 is a chemokine with reported roles in accumulation of leukocyctes during inflammation. CCL13 is also involved in the recruitment of monocytes into the arterial wall during atherosclerosis. In some embodiments, a compound or formulation provided herein can modulate gene and gene products related to CCL7. CCL7 is a chemokine associated with cardiovascular disease, diabetes mellitus, and kidney disease. CCL7 is also reported to play a role in inflammatory events by attracting macrophages and monocytes to further amplify inflammatory processes and contribute to disease progression. In some embodiments, a compound or formulation provided herein can modulate gene and gene products related to CXCL10. CXCL10 binds to CXCR3 and results in pleiotropic effects, including stimulation of monocytes, natural killer, and T-cell migration, and modulation of adhesion molecule expression. CXCL10 may also regulate cytokines storm immune response in response to viral infection, including SARS-CoV-2 infection. In some embodiments, the response to a compound or formulation provided herein can be tracked by evaluating plasma levels and/or gene expression of CCL13, CCL7, and CXCL10 in a subject. In some embodiments, a compound or formulation provided herein can modulate levels of cytokines associated with ALS based on microbial translocation studies.

In some embodiments, a compound or formulation provided herein can modulate levels of a factor (e.g., gene or protein) in HSCs. In some embodiments, a compound or formulation provided herein can modulate levels of a factor (e.g., gene or protein) in HSCs treated with LPS. Non-limiting examples of factors that can be modulated include SMN2, NUDT4P1, TNFSF12, TNFSF13, CXCL10, CCL8, CFB, IF144L, ZN625, ZNF20, PCDHGB6, HST1H4J, MX1, IF127, OAS2, OAS1, IFITI, SIGLECI, CCL7, OAS3, RSAD2, MX2, IFI44, OASL, ISG15, HELZ2, IFI7, IFIT3, XAF1, USP18, RGPD6, CD207, SDCBP2, GBP1P1, PDGFlPL, PDGFRL, MIA-RAB4B, BLOC1S5-TXNDC5, IRF7, IFIT2, ClOorfll1, TBC1D3K, HIST1H2BI, RPS1O-NWDT3, C7orf55-LUC7L2, CKMT1A, FAM66C, RIC3, SYNJ2BP-COX16, CNTF, and UBD.

In some embodiments, a compound or formulation provided herein can modulate the production of stem-cell derived inflammatory cells. In some embodiments, a compound or formulation provided herein can decrease the production of stem-cell derived inflammatory cells. In some embodiments, a compound or formulation provided herein can halt the production of stem-cell derived inflammatory cells. In some embodiments, a compound or formulation provided herein can modulate the differentiation of hematopoietic stem cells (HSCs). In some embodiments, a compound or formulation provided herein can reverse the differentiation of HSCs into inflammatory cells associated with disease progression. In some embodiments, a compound or formulation provided herein modulate the loss of HSC self-replacement efficiency. In some embodiments, a compound or formulation provided herein can modulate the loss of HSC self-replacement efficiency associated with depletion of telomeres. In some embodiments, a compound or formulation provided herein can decrease the loss of HSC self-replacement efficiency. In some embodiments, a compound or formulation provided herein can modulate gene expression in HSCs. In some embodiments, a compound or formulation provided herein can modulate gene expression in HSCs associated with inflammation. In some embodiments, a compound or formulation provided herein can modulate gene expression in HSCs induced by an endotoxin exposure. In some embodiments, a compound or formulation provided herein can decrease gene expression in HSCs induced by endotoxin exposure. Non-limiting examples of gene ontology pathways associated with endotoxin exposure in HSCs include but are not limited to: defense response to symbiont, defense response to virus, cytokine-mediated signaling pathway, response to virus, cytokine production, regulation of immune response, inflammatory response, regulation of response to external stimulus, response to cytokine, innate immune response, regulation of immune system process, defense response to other organism, response to external biotic stimulus, response to other organism, response to biotic stimulus, immune response, biological process involved in interspecies interaction, defense response, immune system process, response to external stimulus, and response to stress.

### Embodiments

The following embodiments are not intended to limit the scope of the invention.

Embodiment 1. A method of reducing a deleterious effect of aging in a subject, the method comprising administering to the subject an effective amount of a chlorite salt, wherein the deleterious effect of aging is not a disease and is not life-threatening.

Embodiment 2. The method of Embodiment 1, wherein the deleterious effect of aging is associated with over-replication of stem cells.

Embodiment 3. The method of any one of Embodiments 1-2, wherein the chlorite salt is sodium chlorite.

Embodiment 4. The method of any one of Embodiments 1-3, wherein the administering comprises a regimen of dosings of the chlorite salt, wherein each dosing comprises administration of a pharmaceutical formulation, wherein the pharmaceutical formulation comprises the chlorite salt and a pharmaceutically-acceptable excipient.

Embodiment 5. The method of Embodiment 4, wherein the pharmaceutical formulation has a pH from 7.0 to 9.5.

Embodiment 6. The method of Embodiment 4, wherein the pharmaceutical formulation has a pH from 7.5 to 9.0.

Embodiment 7. The method of Embodiment 4, wherein the pharmaceutically-acceptable excipient is dibasic sodium phosphate.

Embodiment 8. The method of Embodiment 4, wherein the pharmaceutical formulation is substantially free of preservatives.

Embodiment 9. The method of Embodiment 4, wherein the chlorite salt is present in the pharmaceutical formulation at a concentration of about 62 mM.

Embodiment 10. The method of Embodiment 4, wherein the pharmaceutical formulation has a volume of about 250 mL.

Embodiment 11. The method of any one of Embodiments 1-10, wherein the administering is oral.

Embodiment 12. The method of any one of Embodiments 1-10, wherein the administering is parenteral.

Embodiment 13. The method of any one of Embodiments 1-10 and 12, wherein the administering is by infusion over about 30 to about 60 minutes.

Embodiment 14. The method of any one of Embodiments 1-10 and 12-13, wherein the administering is by infusion over about 30 minutes.

Embodiment 15. The method of any one of Embodiments 1-14, wherein the effective amount is about 2 mg/kg subject body mass of the chlorite salt.

Embodiment 16. The method of any one of Embodiments 1-15, wherein the administering occurs on each of five consecutive days in a first month and on each of three consecutive days in each following month.

Embodiment 17. The method of any one of Embodiments 1-15, wherein the administering of the chlorite salt occurs about every other day.

Embodiment 18. The method of any one of Embodiments 1-15, wherein the administering of the chlorite salt occurs about every day.

Embodiment 19. The method of any one of Embodiments 1-18, further comprising determining by observation that the deleterious effect of aging has been reduced in the subject.

Embodiment 20. The method of any one of Embodiments 1-19, wherein after the administering the chlorite salt reduces the deleterious effect of aging by blocking unnecessary replication of stem cells, thereby slowing the depletion of telomeres in the stem cells, thereby keeping the stem cells active, wherein the chlorite salt blocks the unnecessary replication of stem cells in an inflammatory environment, wherein the inflammatory environment activates chlorite ion to form taurine chloramine.

Embodiment 21. A method comprising: a) administering to a subject an effective amount of a chlorite salt; and b) subsequent to the administering, evaluating telomere length in a stem cell taken from the subject after the administering.

Embodiment 22. The method of Embodiment 21, further comprising taking the stem cell from the subject at least six months after the administering.

Embodiment 23. The method of any one of Embodiments 21-22, wherein the chlorite salt is sodium chlorite.

Embodiment 24. The method of any one of Embodiments 21-23, wherein the administering comprises a regimen of dosings of the chlorite salt, wherein each dosing comprises administration of a pharmaceutical formulation, wherein the pharmaceutical formulation comprises the chlorite salt and a pharmaceutically-acceptable excipient.

Embodiment 25. The method of Embodiment 24, wherein the pharmaceutical formulation has a pH from 7.0 to 9.5.

Embodiment 26. The method of Embodiment 24, wherein the pharmaceutical formulation has a pH from 7.5 to 9.0.

Embodiment 27. The method of Embodiment 24, wherein the pharmaceutically-acceptable excipient is dibasic sodium phosphate.

Embodiment 28. The method of Embodiment 24, wherein the pharmaceutical formulation is substantially free of preservatives.

Embodiment 29. The method of Embodiment 24, wherein the chlorite salt is present in the pharmaceutical formulation at a concentration of about 62 mM.

Embodiment 30. The method of Embodiment 24, wherein the pharmaceutical formulation has a volume of about 250 mL.

Embodiment 31. The method of any one of Embodiments 21-30, wherein the administering is oral.

Embodiment 32. The method of any one of Embodiments 21-30, wherein the administering is parenteral.

Embodiment 33. The method of any one of Embodiments 21-30 and 32, wherein the administering is by infusion over about 30 to about 60 minutes.

Embodiment 34. The method of any one of Embodiments 21-30 and 32-33, wherein the administering is by infusion over about 30 minutes.

Embodiment 35. The method of any one of Embodiments 21-34, wherein the effective amount is about 2 mg/kg subject body mass of the chlorite salt.

Embodiment 36. The method of any one of Embodiments 21-35, wherein the administering occurs on each of five consecutive days in a first month and on each of three consecutive days in each following month.

Embodiment 37. The method of any one of Embodiments 21-35, wherein the administering of the chlorite salt occurs about every other day.

Embodiment 38. The method of any one of Embodiments 21-35, wherein the administering of the chlorite salt occurs about every day.

Embodiment 39. The method of any one of Embodiments 21-38, wherein the evaluating determines that the telomere length is longer than the telomere length would have been had the subject not received the chlorite salt.

Embodiment 40. The method of any one of Embodiments 21-39, further comprising providing additional administrations of the chlorite salt to the subject based at least in part on the telomere length.

Embodiment 41. A method comprising: a) administering to a subject an effective amount of a chlorite salt; and b) subsequent to the administering, monitoring cell division in a stem cell taken from the subject after the administering.

Embodiment 42. The method of Embodiment 41, further comprising taking the stem cell from the subject at least six months after the administering.

Embodiment 43. The method of any one of Embodiments 41-42, wherein the chlorite salt is sodium chlorite.

Embodiment 44. The method of any one of Embodiments 41-43, wherein the administering comprises a regimen of dosings of the chlorite salt, wherein each dosing comprises administration of a pharmaceutical formulation, wherein the pharmaceutical formulation comprises the chlorite salt and a pharmaceutically-acceptable excipient.

Embodiment 45. The method of Embodiment 44, wherein the pharmaceutical formulation has a pH from 7.0 to 9.5.

Embodiment 46. The method of Embodiment 44, wherein the pharmaceutical formulation has a pH from 7.5 to 9.0.

Embodiment 47. The method of Embodiment 44, wherein the pharmaceutically-acceptable excipient is dibasic sodium phosphate.

Embodiment 48. The method of Embodiment 44, wherein the pharmaceutical formulation is substantially free of preservatives.

Embodiment 49. The method of Embodiment 44, wherein the chlorite salt is present in the pharmaceutical formulation at a concentration of about 62 mM.

Embodiment 50. The method of Embodiment 44, wherein the pharmaceutical formulation has a volume of about 250 mL.

Embodiment 51. The method of any one of Embodiments 41-50, wherein the administering is oral.

Embodiment 52. The method of any one of Embodiments 41-50, wherein the administering is parenteral.

Embodiment 53. The method of any one of Embodiments 41-50 and 52, wherein the administering is by infusion over about 30 to about 60 minutes.

Embodiment 54. The method of any one of Embodiments 41-50 and 52-53, wherein the administering is by infusion over about 30 minutes.

Embodiment 55. The method of any one of Embodiments 41-54, wherein the effective amount is about 2 mg/kg subject body mass of the chlorite salt.

Embodiment 56. The method of any one of Embodiments 41-55, wherein the administering occurs on each of five consecutive days in a first month and on each of three consecutive days in each following month.

Embodiment 57. The method of any one of Embodiments 41-55, wherein the administering of the chlorite salt occurs about every other day.

Embodiment 58. The method of any one of Embodiments 41-55, wherein the administering of the chlorite salt occurs about every day.

Embodiment 59. The method of any one of Embodiments 41-58, wherein the monitoring the cell division determines that the cell division occurs at a rate that is lesser than what the rate would have been had the subject not received the chlorite salt.

Embodiment 60. The method of any one of Embodiments 41-59, further comprising providing additional administrations of the chlorite salt to the subject based at least in part on the monitoring the cell division.

Embodiment 61. A method comprising: a) administering to a subject an effective amount of a chlorite salt; and b) subsequent to the administering, monitoring the subject for depletion of a telomere of a stem cell of the subject.

Embodiment 62. The method of Embodiment 61, further comprising taking the stem cell from the subject at least six months after the administering.

Embodiment 63. The method of any one of Embodiments 61-62, wherein the chlorite salt is sodium chlorite.

Embodiment 64. The method of any one of Embodiments 61-63, wherein the administering comprises a regimen of dosings of the chlorite salt, wherein each dosing comprises administration of a pharmaceutical formulation, wherein the pharmaceutical formulation comprises the chlorite salt and a pharmaceutically-acceptable excipient.

Embodiment 65. The method of Embodiment 64, wherein the pharmaceutical formulation has a pH from 7.0 to 9.5.

Embodiment 66. The method of Embodiment 64, wherein the pharmaceutical formulation has a pH from 7.5 to 9.0.

Embodiment 67. The method of Embodiment 64, wherein the pharmaceutically-acceptable excipient is dibasic sodium phosphate.

Embodiment 68. The method of Embodiment 64, wherein the pharmaceutical formulation is substantially free of preservatives.

Embodiment 69. The method of Embodiment 64, wherein the chlorite salt is present in the pharmaceutical formulation at a concentration of about 62 mM.

Embodiment 70. The method of Embodiment 64, wherein the pharmaceutical formulation has a volume of about 250 mL.

Embodiment 71. The method of any one of Embodiments 61-70, wherein the administering is oral.

Embodiment 72. The method of any one of Embodiments 61-70, wherein the administering is parenteral.

Embodiment 73. The method of any one of Embodiments 61-70 and 72, wherein the administering is by infusion over about 30 to about 60 minutes.

Embodiment 74. The method of any one of Embodiments 61-70 and 72-73, wherein the administering is by infusion over about 30 minutes.

Embodiment 75. The method of any one of Embodiments 61-74, wherein the effective amount is about 2 mg/kg subject body mass of the chlorite salt.

Embodiment 76. The method of any one of Embodiments 61-75, wherein the administering occurs on each of five consecutive days in a first month and on each of three consecutive days in each following month.

Embodiment 77. The method of any one of Embodiments 61-75, wherein the administering of the chlorite salt occurs about every other day.

Embodiment 78. The method of any one of Embodiments 61-75, wherein the administering of the chlorite salt occurs about every day.

Embodiment 79. The method of any one of Embodiments 61-78, wherein the monitoring the subject for depletion of the telomere of the stem cell of the subject determines that the depletion occurs at a rate that is lesser than what the rate would have been had the subject not received the chlorite salt.

Embodiment 80. The method of any one of Embodiments 61-79, further comprising providing additional administrations of the chlorite salt to the subject based at least in part on the monitoring the subject for depletion of the telomere of the stem cell of the subject.

Embodiment 81. A method comprising: a) administering to a subject an effective amount of a chlorite salt; and b) subsequent to the administering, monitoring the subject for replication of stem cells.

Embodiment 82. The method of Embodiment 81, further comprising monitoring the subject for replication of stem cells at least six months after the administering.

Embodiment 83. The method of any one of Embodiments 81-82, wherein the chlorite salt is sodium chlorite.

Embodiment 84. The method of any one of Embodiments 81-83, wherein the administering comprises a regimen of dosings of the chlorite salt, wherein each dosing comprises administration of a pharmaceutical formulation, wherein the pharmaceutical formulation comprises the chlorite salt and a pharmaceutically-acceptable excipient.

Embodiment 85. The method of Embodiment 84, wherein the pharmaceutical formulation has a pH from 7.0 to 9.5.

Embodiment 86. The method of Embodiment 84, wherein the pharmaceutical formulation has a pH from 7.5 to 9.0.

Embodiment 87. The method of Embodiment 84, wherein the pharmaceutically-acceptable excipient is dibasic sodium phosphate.

Embodiment 88. The method of Embodiment 84, wherein the pharmaceutical formulation is substantially free of preservatives.

Embodiment 89. The method of Embodiment 84, wherein the chlorite salt is present in the pharmaceutical formulation at a concentration of about 62 mM.

Embodiment 90. The method of Embodiment 84, wherein the pharmaceutical formulation has a volume of about 250 mL.

Embodiment 91. The method of any one of Embodiments 81-90, wherein the administering is oral.

Embodiment 92. The method of any one of Embodiments 81-90, wherein the administering is parenteral.

Embodiment 93. The method of any one of Embodiments 81-90 and 92, wherein the administering is by infusion over about 30 to about 60 minutes.

Embodiment 94. The method of any one of Embodiments 81-90 and 92-93, wherein the administering is by infusion over about 30 minutes.

Embodiment 95. The method of any one of Embodiments 81-94, wherein the effective amount is about 2 mg/kg subject body mass of the chlorite salt.

Embodiment 96. The method of any one of Embodiments 81-95, wherein the administering occurs on each of five consecutive days in a first month and on each of three consecutive days in each following month.

Embodiment 97. The method of any one of Embodiments 81-95, wherein the administering of the chlorite salt occurs about every other day.

Embodiment 98. The method of any one of Embodiments 81-95, wherein the administering of the chlorite salt occurs about every day.

Embodiment 99. The method of any one of Embodiments 81-98, wherein the monitoring the subject for replication of stem cells determines that the replication occurs at a rate that is lesser than what the rate would have been had the subject not received the chlorite salt.

Embodiment 100. The method of any one of Embodiments 81-99, further comprising providing additional administrations of the chlorite salt to the subject based at least in part on the monitoring the subject for replication of stem cells.

### EXAMPLES

The following examples are provided for illustrative purposes only and not to limit the scope of the claims provided herein.

### Example 1: Therapeutic Effects of NP001

A series of experiments are designed to determine the therapeutic effects of sodium chlorite on clinical biomarkers and microbial translocation in ALS patients.

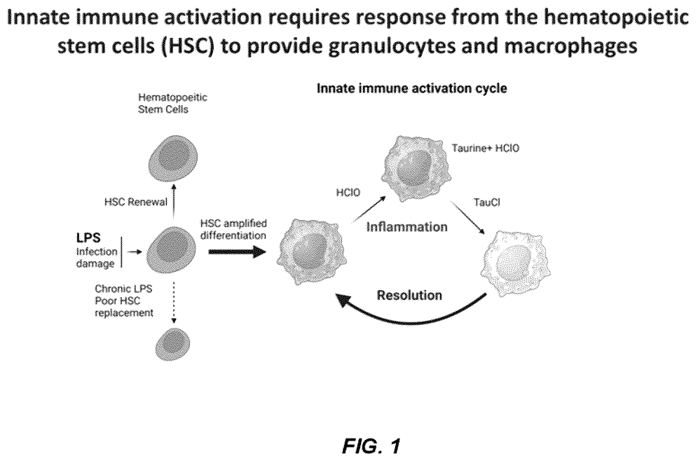

The therapeutic effects of NP001, a form of sodium chlorite for intravenous injection, are dependent on endogenous stem cells. NP001 exhibits therapeutic effects in hematopoietic stem cells. In mechanism of action studies in patients with ALS, NP001 was observed to restore innate immune function while slowing disease progression. In a normal immune response, HSCs can reproduce while providing differentiated cells in response to insults such as infection and tissue damage. In the face of chronic activation mediated by LPS, which characterizes the innate immune system in ALS, the HSC can gradually lose self-replacement efficiency, for example, by depletion of telomeres. FIG. 1 illustrates the loss of HSC renewal in situations with chronic innate immune system activation.

Evaluation of Clinical Biomarkers in ALS Patients after NP001 Administration.

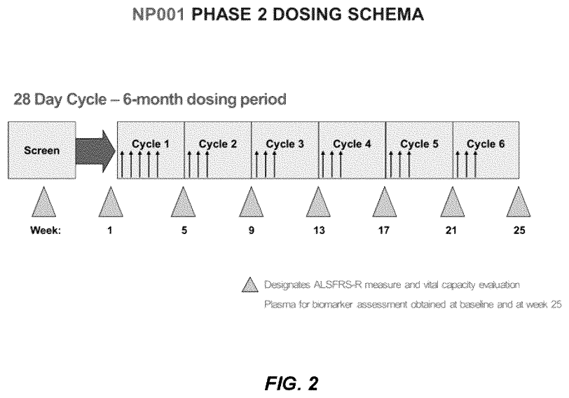

NP001 is administered as a pulse therapy as a 30′ infusion 5 days in a row the first month followed by 3 days in a row per month thereafter (FIG. 2). Clinical phase 2 biomarkers are measured in patients with clinical responses to NP001. Non-limiting examples of measures of clinical response include the ALS Functional Rating Scale Revised (ALSFRS-R) and respiratory vital capacity. Clinical response and biomarkers are measured as changes from baseline after treatment (e.g., six months of NP001). The mechanism of action of NP001 is determined through measuring biomarkers, including biomarkers of HSC activation. Biomarker levels are tracked after NP001 administration for a long term period of time (e.g., 6 months or 1 year) to examine long term changes after NP001 pulse therapy.

### Effect of NP001 on Microbial Translocation

Patients with neurodegenerative diseases such as ALS show evidence for ongoing microbial translocation with elevated plasma levels of the immune activator LPS. Innate immune activation in ALS is driven by microbial translocation associated LPS with LPS affecting the rate at which the innate immune response occurs, but also results in differentiation of stem cells into cells capable of driving the inflammatory response and suppressing myeloid stem cell function.

Plasma levels of LPS are elevated in ALS with blood monocytes expressing LPS-activated genes proportional to plasma LPS levels, indicating that immunologic dysfunction in ALS is driven by microbial translocation. Microbial translocation in ALS is posited to occur via the following mechanism: (1) ALS progression advances to the point wherein abnormal neurons stimulate local microglia to produce proinflammatory factors which activate blood monocytes to become proinflammatory; (2) Inflammatory macrophages do not phagocytose microorganisms or participate in wound healing. The epithelial cells of the colon turn over every 5 days, making the wound healing function important for maintaining colonic epithelium integrity. The leakage of bacteria/bacterial products occurs in the blood; (3) Bacterial endotoxins (LPS) activate blood monocytes and tissue macrophages, leading to a systemic proinflammatory state; and (4) Monocytes are recruited from the blood into damaged tissues to initiate repair. However, LPS activation leads to trafficking of non-wound healing, non-phagocytic, proinflammatory cells into the damaged tissues, leading to persistence of the disease.

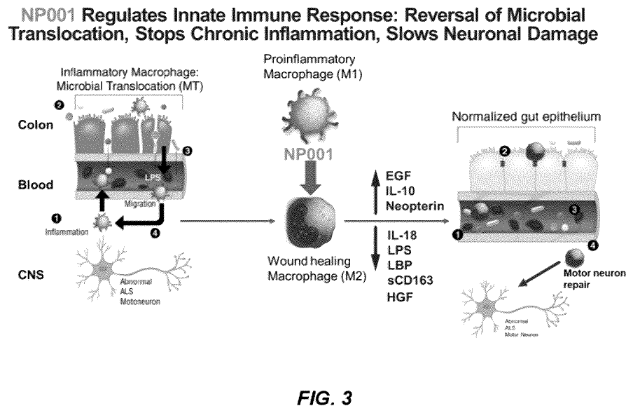

NP001 is administered to patients in a pulse therapy administration procedure. Following administration of NP001, levels of plasma biomarkers are measured in patients who show clinical response using ALSFRS-R and respiratory vital capacity score changes in treated and control arms at 6 months of administration. Stem cell reprogramming for long term disease regulation is evidenced by finding normalization of biomarkers for at least one month after the last dose in the 6-month administration procedure. Long term changes in 8 plasma biomarkers: upregulation of epidermal growth factor (EGF), interleukin-10 (IL-10), neopterin, and downregulation of interleukin-18 (IL-18), lipopolysaccharide (LPS), lipopolysaccharide binding protein (LBP), soluble CD163 (sCD163), and hepatocyte growth factor (HGF), shows reversal of microbial translocation-associated immune activation by NP001. The reversal of microbial translocation-associated immune activation by NP001 is illustrated in FIG. 3. NP001 regulates of microbial translocation by removing LPS-driven inflammation mediated repression of myeloid precursor functions. NP001 regulation of microbial translocation occurs via the following mechanism (1) NP001-induced taurine chloramine (TauCl) downregulates NFkB and proinflammatory factors; (2) increased EGF augments gut epithelium repair. Bacterial translocation resolves; (3) macrophages are converted to be phagocytic and wound healing; and (4) a drop in sCD163 signifies that blood monocyte traffic to CNS is decreased. NP001 treatment leads to the resolution of biomarker levels associated with the microbial translocation process and resolves macrophage dysfunction. Given the infrequent dosing of NP001 and the short half-life of minutes, any prolonged activity at regulating the innate immune system extending beyond the brief time the drug is present in vivo (half-life minutes) suggests that activities that extend beyond reversing activation of short-lived granulocytes and blood derived macrophages.

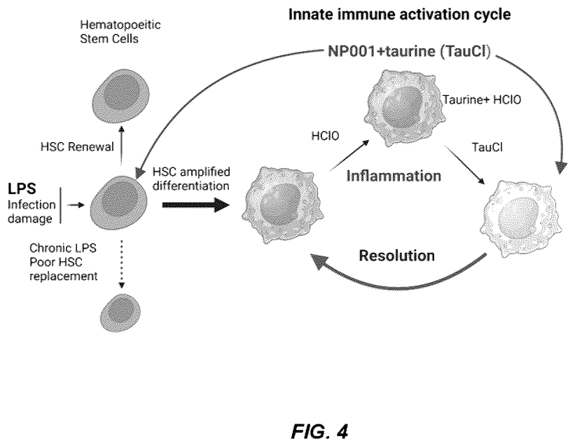

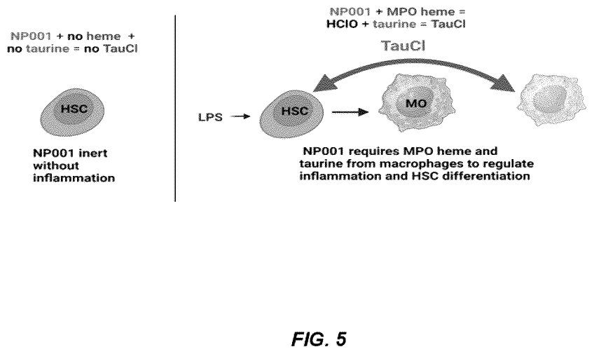

NP001 regulates HSC function and the innate immune system in patients with ongoing inflammation by reversing macrophage activation and stimuli that cause HSCs to make inflammatory hematopoietic cells that inappropriately drive ALS pathogenesis. In patients with ALS, the activation stimulus is LPS, a factor also implicated in driving HSCs to differentiate. The long-term effects of NP001 at regulating the innate immune system are through the action at reversing HSC differentiation into inflammatory cells that inappropriately drive disease progression. The effect of NP001 on innate immune activation and dysfunctional immune response is illustrated in FIG. 4. FIG. 5 illustrates reversal of LPS-activated HSC genetic pathways by a single dose of NP001 (MPO=myeloperoxidase)

### Example 2: Effect of Sodium Chlorite on HSC Activation

A series of experiments were designed to determine whether sodium chlorite restores innate immune function through the regulation of HSC activation.

A major subset of ALS patients show evidence for ongoing inflammation that potentially contributes to disease progression. NP001 is a form of sodium chlorite for intravenous injection. In phase 2 and follow up biomarker mechanism of action (MOA) studies in patients with ALS, NP001 was observed to restore innate immune function while slowing disease progression in patients with plasma CRP levels >1.13 mg/L. Intermittent dosing (3 days/month) showed prolonged regulation of 8 biomarkers associated with microbial translocation in patients whose disease slowed. Microbial translocation is observed to be a major stimulus for HSC to produce inflammatory cells, a process that contributes to ongoing innate immune dysfunction and gradual depletion of HSC reservoirs in vivo.

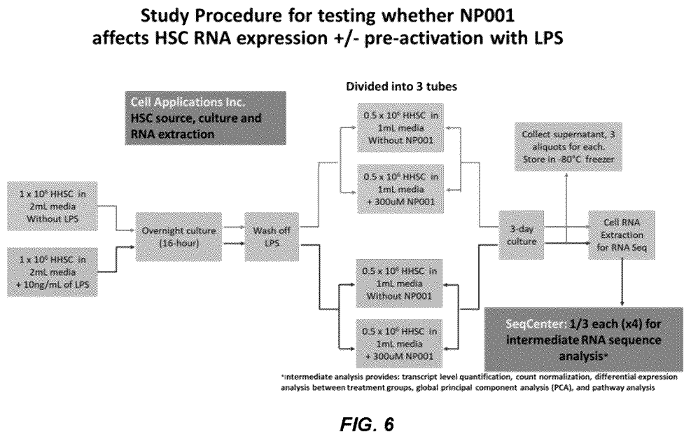

CD 34+ HSCs were isolated from normal human adult bone marrow (>95% pure) and were either stimulated by 10 ng/mL LPS overnight or control medium. NP001 was added (300 μM; equivalent to phase 2 dose in humans) to washed HSC controls and LPS-activated HSC and the cells grown for 3 days in HSC medium. RNAseq with 100 M reads was performed on all cells and pathway analysis was performed. A summary of the experimental methods is detailed in FIG. 6.

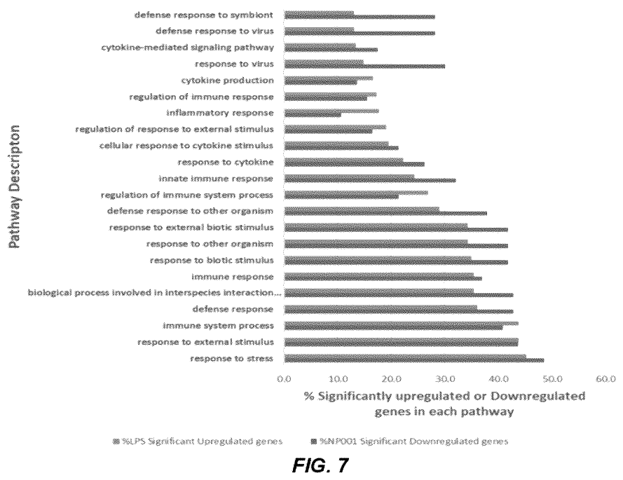

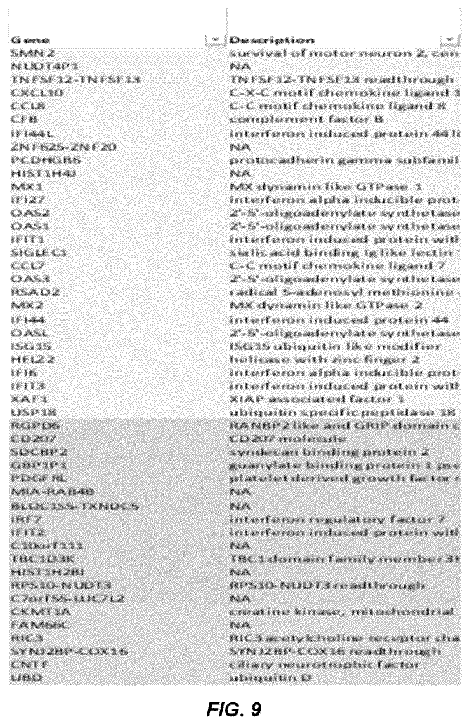

LPS activation of HSC caused expression of genes involved in innate immune responses. By comparison with non-LPS-activated cells, LPS-activated pathways with p<10-15 were defined. The pathways were mostly defensive vs organisms, viruses, and bacteria, and involved activation of innate immune response and inflammatory genes (FIGS. 7 & 8). NP001-treated, LPS-activated HSC gene expression pathways were evaluated in parallel. Without exception, the HSC pathways stimulated by LPS were reversed after a single treatment with NP001. Parallel NP001 treatment of HSC controls showed no activity. In addition, LPS-activated HSC were evaluated for factors (e.g., protein and genes), that were modulated following treatment with NP001. A summary of the factors is detailed in FIG. 9.

LPS activation plays a role in both ALS pathogenesis and depletion over time of HSC function. HSC activation and NP001 treatment in the current study allows some understanding as to why NP001-responsive patients are those with underlying innate immune activation. This activation converts NP001 from a prodrug to one that produces a long-acting regulator of inflammation, taurine chloramine (TauCl). TauCl also acts at the HSC level, feeding back on activation pathways induced by LPS. A theory that explains these observations is that NP001 acts to reverse immune activation by neutralizing the inflammatory phenotype and to halt production of HSC-derived inflammatory cells while showing no observed side effects either in HSC cultures or in patients receiving NP001 who expressed LPS activation pathways. No NP001 activity was observed in non-stimulated HSC cultures.

### Example 3: Evaluation of Response to NP001

Response to NP001 can be evaluated in a subject or in HSCs obtained from a subject can be evaluated by examining levels of chemokines and cytokines. Plasma levels or expression levels of genes related to CCL13, CCL7, and CXCL10 are measured in a subject or in HSCs obtained from a subject prior to the administration of NP001. NP001 is administered according to pulse therapy administration procedure detailed in Example 1. Following administration of NP001, plasma levels or gene expression of CCL13, CCL7, and CXCL10 are measured in the same subject or in HSCs obtained from a subject to evaluate the effect of NP001 treatment. Accordingly, CCL13, CCL7, and CXCL10 can serve as a diagnostic or theragnostic for NP001 administration.

1.-20. (canceled)

21. A method comprising: a) administering to a subject an effective amount of a chlorite salt; and b) subsequent to the administering, evaluating telomere length in a stem cell taken from the subject after the administering.

22. The method of claim 21, further comprising taking the stem cell from the subject at least six months after the administering.

23. The method of claim 21, wherein the chlorite salt is sodium chlorite.

24. The method of claim 21, wherein the administering comprises a regimen of dosings of the chlorite salt, wherein each dosing comprises administration of a pharmaceutical formulation, wherein the pharmaceutical formulation comprises the chlorite salt and a pharmaceutically-acceptable excipient.

25. The method of claim 24, wherein the pharmaceutical formulation has a pH from 7.0 to 9.5.

26. The method of claim 24, wherein the pharmaceutical formulation has a pH from 7.5 to 9.0.

27. The method of claim 24, wherein the pharmaceutically-acceptable excipient is dibasic sodium phosphate.

28. The method of claim 24, wherein the pharmaceutical formulation is substantially free of preservatives.

29. The method of claim 24, wherein the chlorite salt is present in the pharmaceutical formulation at a concentration of about 62 mM.

30. The method of claim 24, wherein the pharmaceutical formulation has a volume of about 250 mL.

31. The method of claim 21, wherein the administering is oral.

32. The method of claim 21, wherein the administering is parenteral.

33. The method of claim 21, wherein the administering is by infusion over about 30 to about 60 minutes.

34. The method of claim 21, wherein the administering is by infusion over about 30 minutes.

35. The method of claim 21, wherein the effective amount is about 2 mg/kg subject body mass of the chlorite salt.

36. The method of claim 21, wherein the administering occurs on each of five consecutive days in a first month and on each of three consecutive days in each following month.

37. The method of claim 21, wherein the administering of the chlorite salt occurs about every other day.

38. The method of claim 21, wherein the administering of the chlorite salt occurs about every day.

39. The method of claim 21, wherein the evaluating determines that the telomere length is longer than the telomere length would have been had the subject not received the chlorite salt.

40. The method of claim 21, further comprising providing additional administrations of the chlorite salt to the subject based at least in part on the telomere length.

41.-100. (canceled)
